(19) United States
(12) Patent Application Publication
(10) Pub. No.: US 2003/0232388 A1
(43) Pub. Date: Dec. 18, 2003

(54) Beads having identifiable Raman markers

(76) Inventors: David I. Kreimer, Berkeley, CA (US); Lev Ginzburg, Fremont, CA (US); Thomas H. Nufert, Walnut Creek, CA (US); Oleg A. Yevin, Oakland, CA (US)

Correspondence Address: FLIESLER DUBB MEYER & LOVEJOY, LLP, FOUR EMBARCADERO CENTER, SUITE 400, SAN FRANCISCO, CA, 94111, US

(21) Appl. No.: 10/298,725

(22) Filed: Nov. 18, 2002

Related U.S. Application Data
(63) Continuation-in-part of application No. 09/925,189, filed on Aug. 8, 2001.
(63) Continuation-in-part of application No. 09/815,909, filed on Mar. 23, 2001.
(63) Continuation-in-part of application No. 09/670,453, filed on Sep. 26, 2000.
(60) Provisional application No. 60/333,303, filed on Nov. 18, 2001.
(60) Provisional application No. 60/156,195, filed on Sep. 27, 1999.

(51) Int. Cl. G01N 33/53; G01N 33/553

(52) U.S. Cl. 435/7.1; 436/525

(57) ABSTRACT

This invention comprises novel enhancing particle structures and beads which can have receptor molecules attached thereto. The structures are useful for Raman spectroscopic detection of markers associated with analyses of analytes in complex solutions containing molecules of interest. Analytes that can be detected using these methods include nucleic acids, proteins, cytokines, hormones, vitamins, those from bacteria, viruses, cells and tissues, and other molecules that can specifically bind to the analyte receptors. Beads can be used as biomarkers, as analytical tools, and as tags for combinatorial syntheses.

### RELATED APPLICATIONS

This application claims priority to U.S. Application No. 60/333,303, filed Nov. 18, 2001, and is a continuation-in-part of U.S. application Ser. No. 09/925,189, filed Aug. 8, 2001, which is a continuation-in-part of U.S. application Ser. No. 09/815,909, which is a continuation-in-part of U.S. application Ser. No. 09/670,453, which claimed priority to U.S. provisional application Serial No. 60/156,195 filed Sep. 27, 1999. Each of these Patent Applications is herein incorporated fully by reference.

### BACKGROUND OF THE INVENTION

1. Field of the Invention

This invention relates to the manufacture of beads having identifiable markers thereon. Specifically, the invention relates to beads having identifiable Raman markers. More specifically, the invention relates beads having identifiable Raman markers, Raman enhancing structures and analyte receptors.

2. Description of Related Art

Numerous biological, analytical and synthetic methods use small solid substrates, or “beads” attached to which are moieties having identifiable characteristics. Such identifiable characteristics include fluorescence, radiofrequency production, Raman scattering, absorption, Raleigh scattering, radioactivity, spin resonance, magnetic resonance, and mass/charge characteristics. Of these, several characteristics are relatively easy and convenient to detect and to measure. Those include fluorescence, absorption, radioactivity, and the like.

Such beads are used to label cells, to be solid supports for molecular syntheses, such as in combinatorial chemistry, and for analyte detection. It can be highly desirable to provide a marker signal that can be easily distinguished from other moieties present. For fluorescence detection of analytes, it may be necessary for the identifiable marker to have a different characteristic from that for the analyte to be detected. Thus, because of potential overlapping signals, it can be limiting to use fluorescent markers in analyses that use fluorescence detection of analytes.

I. Detection of Analytes

The detection and quantification of molecules or “analytes” in complex mixtures containing small amounts of analyte and large numbers and amounts of other materials is a continuing challenge. As more interest is focused upon the roles of biological molecules in physiology and disease processes, the rapid accurate detection of biological molecules is becoming more important.

The detection of analyte, or “ligand” molecules is an important aspect of current biology, biotechnology, chemistry, and environmental industries. Detection of ligands can be accomplished using many different methods, including the chemical methods of chromatography, mass spectroscopy, nucleic acid hybridization and immunology. Hybridization and immunological methods rely upon the specific binding of ligands to detector, or “receptor” molecules. The basis for specificity of these methods is conferred by a receptor molecule can bind in a specific fashion to the ligand molecule, thereby creating a bound complex. Upon treating the complex under conditions that favor the removal of unbound ligand, the bound ligand can be assayed. The specificity of the binding, the completeness of separating bound and unbound ligands and receptors, and the sensitivity of the detection of the ligand confers the selectivity of the detection system. For example, in biology and biotechnology industries, analytes such as deoxyribonucleic acid (“DNA”) and messenger ribonucleic acid (“mRNA”) are important indicators of specific genetic, physiological or pathological conditions. DNA can contain important information about the genetic makeup of an organism, and mRNA can be an important indicator of which genes are active in a specific physiological or pathological condition and what proteins may be created as a result of gene activation. Additionally, the direct detection of proteins can be important to the understanding of the physiological or pathological condition of an individual.

A. Hybridization Detection of Nucleic Acids

Many different methods are currently in use for the detection of nucleic acids and proteins, but those methods can be time-consuming, expensive, or poorly reproducible. For example, the detection of specific nucleic acid sequences in DNA or RNA molecules can be accomplished using hybridization reactions, wherein an analyte DNA or RNA molecule is permitted to attach to a complementary sequence of DNA. A complementary DNA molecule can be attached to a supporting matrix, and the bound DNA and matrix is herein termed a “substrate.” Exposing an analyte nucleic acid to a complementary substrate DNA can result in the formation of a relatively stable hybrid. Detection of the duplex DNA hybrid is characteristically carried out using methods that can detect labeled DNA analytes. The labeling is typically performed using radioactive, spin resonance, chromogenic or other labels, which are attached to the analyte molecules. Thus, when the labeled analyte attaches to the substrate, unbound analyte can be removed and the bound, or specific, analyte can be detected and quantified.

For example, to detect a mRNA molecule having a specific sequence using current methods, naturally occurring, or “native” mRNA is typically converted to a complementary DNA (“cDNA”) molecule using an enzyme called “reverse transcriptase” under conditions that incorporate a labeled nucleotide into the cDNA. Upon binding of the labeled cDNA to the hybridization substrate, the bound ligand can be detected using a radiometric technique such as scintillation counting, fluorescence or spin resonance, depending on the type of label used.

Currently available methods for the detection of nucleic acids and proteins have undesirable characteristics. The methods are time consuming, require expensive equipment and reagents, require expert manual operations, and the reagents can be environmentally hazardous. Additionally, for assaying mRNA, the methods also can be sensitive to defects in the fidelity of reverse transcription. Unless the cDNA made during reverse transcription is exactly complementary to the mRNA, the analyte will not have the same sequence as the native mRNA, and misleading results can be obtained. The amplification of nucleic acid sequences by the polymerase chain reaction (“PCR”) has been used to increase the numbers of nucleic acid molecules (complementary DNA or “cDNA”) that can be detected. PCR requires DNA polymerase enzymes to amplify the cDNA. Some DNA polymerases can insert incorrect bases into a growing strand of newly synthesized cDNA. In addition, the recognition of ceratin cDNA by DNA polymerase and primers used for PCR can vary depending on the specific sequences of DNA in the sample to be amplified. This variation can result in non-proportional amplification of different cDNA molecules. Subsequent amplification of an strand having an incorrect sequence can result in the presence of several different cDNA sequences in the same sample. Thus, the accuracy and sensitivity of analysis of cDNA using PCR can be compromised.

Additionally, for medical diagnostic or forensic purposes, it can be very important for results of tests to be available rapidly. Commonly used methods for detection of specific nucleic acid sequences can be too slow for therapeutic or forensic uses. Thus, there is a need for rapid, accurate measurement of nucleic acid sequences.

B. Detection of Molecules Using Antibodies and Other Binding Partners

Many methods currently exist for analysis of molecules using antibodies, antibody fragments, and molecules that mimic antibodies. Broadly, any molecule that binds to an analyte with sufficient specificity may be used as in a detection system. Such binding molecules are herein referred to as “analyte receptors.” For example, numerous antibodies are available which react relatively selectively, or specifically with a molecule of interest. When a ligand associates with, or binds to an antibody, a complex can be formed. If the analyte is fluorescent, or has an attached fluorescent tag or label (“fluorophore”), then the complex may be detected by observing a fluorescent signal generated by the fluorophore. It can be appreciated that in addition to fluorescent methods, other methods for analyte detection using antibodies are available.

In addition to antibodies, other proteins may be used to detect molecules. For example, detection of cytokines maybe carried out using cytokine receptors. Other molecules may be detected using lectins. Several of these receptors are commercially available. Moreover, one can use Raman scattering as a basis for detecting and quantifying analytes. U.S. patent application Ser. No. 09/670,453 describes the use of Raman methods for analyte detection.

II. Raman Spectroscopy

Raman spectroscopy involves the use of electromagnetic radiation to generate a signal in an analyte molecule. Raman spectroscopic methods have only recently been developed to the point where necessary sensitivity is possible. Raman spectroscopic methods and some ways of increasing the sensitivity of Raman spectroscopy are described herein below.

A. Raman Scattering

According to a theory of Raman scattering, when incident photons having wavelengths in the near infrared, visible or ultraviolet range illuminate a certain molecule, a photon of that incident light can be scattered by the molecule, thereby altering the vibrational state of the molecule to a higher or a lower level. The vibrational state of a molecule is characterized by a certain type of stretching, bending, or flexing of the molecular bonds. The molecule can then spontaneously return to its original vibrational state. When the molecule returns to its original vibrational state, it can emit a characteristic photon having the same wavelength as the incident photon. The photon can be emitted in any direction relative to the molecule. This phenomenon is termed “Raleigh Light Scattering.”

A molecule having an altered vibrational state can return to a vibrational state different from the original state after emission of a photon. If a molecule returns to a state different from the original state, the emitted photon can have a wavelength different from that of the incident light. This type of emission is known as “Raman Scattering” named after C. V. Raman, the discoverer of this effect. If, a molecule returns to a higher vibrational level than the original vibrational state, the energy of the emitted photon will be lower (i.e., have longer wavelength) than the wavelength of the incident photon. This type of Raman scattering is termed “Stokes-shifted Raman scattering.” Conversely, if a molecule is in a higher vibrational state, upon return to the original vibrational state, the emitted photon has a lower energy (i.e., have a shorter wavelength). This type of Raman scattering is termed “anti-Stokes-shifted Raman scattering.” Because many more molecules are in the original state than in an elevated vibrational energy state, typically the Stokes-shifted Raman scattering will predominate over the anti-Stokes-shifted Raman scattering. As a result, the typical shifts of wavelength observed in Raman spectroscopy are to longer wavelengths. Both Stokes and anti-Stokes shifts can be quantitized using a Raman spectrometer.

B. Resonance Raman Scattering

When the wavelength of the incident light is at or near the frequency of maximum absorption for that molecule, absorption of a photon can elevate both the electrical and vibrational states of the molecule. The efficiency of Raman scattering of these wavelengths can be increased by as much as about 10⁸ times the efficiency of wavelengths substantially different from the wavelength of the absorption maximum. Therefore, upon emission of the photon with return to the ground electrical state, the intensity of Raman scattering can be increased by a similar factor.

C. Surface Enhanced Raman Scattering

When Raman active molecules are excited near to certain types of metal surfaces, a significant increase in the intensity of the Raman scattering can be observed. The increased Raman scattering observed at these wavelengths is herein termed “surface enhanced Raman scattering.” The metal surfaces that exhibit the largest increase in Raman intensity comprise minute or nanoscale rough surfaces, typically coated with minute metal particles. For example, nanoscale particles such as metal colloids can increase intensity of Raman scattering to about 10⁶ times or greater, than the intensity of Raman scattering in the absence of metal particles. This effect of increased intensity of Raman scattering is termed “surface enhanced Raman scattering.”

The mechanism of surface enhanced Raman scattering is not known with certainty, but one factor can affect the enhancement. Electrons can typically exhibit a vibrational motion, termed herein “plasmon” vibration. Particles having diameters of about 1/10^{th} the wavelength of the incident light can contribute to the effect. Incident photons can induce a field across the particles, and thereby can alter the movement of mobile electrons in the metal. As the incident light cycles through its wavelength, the induced motion of electrons can follow the light cycles, thereby creating an oscillation of the electron within the metal surface having the same frequency as the incident light. The electrons' motion can produce a mobile electrical dipole within the metal particle. When the metal particles have certain configurations, incident light can cause groups of surface electrons to oscillate in a coordinated fashion, thereby causing constructive interference of the electrical field so generated, creating an area herein termed a “resonance domain.” The enhanced electric field due to such resonance domains therefore can increase the intensity of Raman scattering and thereby can increase the intensity of the signal detected by a Raman spectrometer.

The combined effects of surface enhancement and resonance on Raman scattering is termed “surface enhanced resonance Raman scattering.” The combined effect of surface enhanced resonance Raman scattering can increase the intensity of Raman scattering by about 10¹⁴ or more. It should be noted that the above theories for enhanced Raman scattering may not be the only theories to account for the effect. Other theories may account for the increased intensity of Raman scattering under these conditions.

D. Raman Methods for Detection of Nucleic Acids and Proteins

Several methods have been used for the detection of nucleic acids and proteins. Typically, an analyte molecule can have a reporter group added to it to increase the ability of an analytical method to detect that molecule. Reporter groups can be radioactive, flourescent, spin labeled, and can be incorporated into the analyte during synthesis. For example, reporter groups can be introduced into cDNA made from mRNA by synthesizing the DNA from precursors containing the reporter groups of interest. Additionally, other types of labels, such as rhodamine or ethidium bromide can intercalate between strands of bound nucleic acids in the assay and serve as reporter groups of hybridized nucleic acid oligomers.

In addition to the above methods, several methods have been used to detect nucleic acids using Raman spectroscopy. Vo-Dinh, U.S. Pat. No. 5,814,516; Vo-Dinh, U.S. Pat. No. 5,783,389; Vo-Dinh, U.S. Pat. No. 5,721,102; Vo-Dinh, U.S. Pat. No. 5,306,403. These patents are herein incorporated fully by reference. Recently, Raman spectroscopy has been used to detect proteins. Tarcha et al., U.S. Pat. No. 5,266,498; Tarcha et al., U.S. Pat. No. 5,567,628, both incorporated herein fully by reference, provide an analyte that has been labeled using a Raman active label and an unlabeled analyte in the test mixture. The above-described methods rely upon the introduction of a Raman active label, or “reporter” group, into the analyte molecule. The reporter group is selected to provide a Raman signal that is used to detect and quantify the presence of the analyte.

More recently, U.S. patent application Ser. No. 09/670,453 described the use of Raman methods that can be used without the need for providing an added Raman reporter group. The methods described provide nanoparticle structures that can act as enhancing structures, which increase the magnitude of a Raman signal generated by a molecule near or attached to the nanoparticle structure.

However, it can be highly desirable to provide a means for labeling or marking individual beads or positions on a substrate with a label that can be easily and reliably discriminated from background signals and from the signal of the receptor molecule, the analyte molecule to be detected and from other beads. Moreover, it is desirable to provide means for marking large numbers of beads individually, for use for either biological, analytical or combinatorial synthetic purposes.

### SUMMARY OF THE INVENTION

Compositions useful for marking beads of the present invention can use particle structures that are designed to enhance Raman signals. Particle structures may be fractal, random or ordered, and may be placed near to or linked with Raman markers having a characteristic Raman spectral feature.

In certain embodiments of this invention, particle structures can be generated using chemical methods using linkers to produce pairs of enhancing particles or larger groups of particles. Such linked particle structures can be designed and manufactured to have desired properties, including but not limited to increased mechanical strength and/or selection of wavelengths of incident electromagnetic radiation that permit the generation of enhanced Raman signals to permit sensitive detection of a variety of analytes.

The enhancing structures are desirably close to a moiety that generates an identifiable Raman signal, and when the enhancing particle and the Raman marker are close together, the intensity of the Raman signal can be substantially increased. The Raman marker may be attached to the enhancing particle directly or indirectly using a bridging moiety. Alternatively, the Raman marker maybe attached to the linker that joins pairs of enhancing particles together. In other embodiments, Raman markers may be associated with multiple areas in the particle groups.

In certain embodiments, enhancing particles and Raman markers can be used in isolation, as biomarkers to localize certain cell types within a body or tissue. Antibodies, lectins and hormone or cytokine receptors as well as a variety of other types of analyte receptors can be used for that purpose.

In other embodiments, enhancing particles with Raman markers can be placed in the interior of a bead having analyte receptors thereon. The beads can then be used to bind to certain analytes and subsequently analyzed for the presence of the analyte. In certain embodiments, it can be desirable to separate the Raman marker from the analyte receptor, so that interference (e.g., quenching) of either signal does not interfere with the detection process. It can be especially useful to separate fluorescence detection of analytes on the surface of the bead, while the Raman marker signal is within the interior of the bead, sufficiently far from the analyte molecule being detected so that fluorescence of the analyte is not quenched.

In certain other embodiments, an analyte receptor can have a Raman marker attached, providing for identification of the bead and analyte binding by the same molecule. Additionally, the amount of Raman marker present on a bead or on another type of substrate can be a measure of the number of receptor molecules present.

By using a number of different Raman markers, one can provide a very large number of combinations of markers, permitting the labeling and identification of a large number of different beads.

The Raman labeled beads of this invention can be used for identification of individual beads in assays in which each bead has a different type of analyte receptor. Additionally, using the Raman labeled beads of this invention, one can detect cells, tissues, and/or pathogens based on the specificity of receptors, and then determine the presence of those beads based on Raman spectroscopy. Additionally, one can carry out combinatorial syntheses and have readily identifiable markers to characterize and select individual synthetic molecules associated with those beads.

### BRIEF DESCRIPTION OF THE DRAWINGS

The invention will be described with respect to the particular embodiments thereof. Other objects, features, and advantages of the invention will become apparent with reference to the specification and drawings in which:

FIG. 1a is a drawing depicting a prior art bead having a single, spherical Raman enhancing particle therein and having Raman markers on the enhancing particle.

FIG. 1b is a drawing depicting a bead of this invention having a rod-shaped Raman enhancing particle therein and Raman markers on the enhancing particle.

FIG. 1c is a drawing depicting a bead of this invention having a crystal-shaped Raman enhancing particle therein and Raman markers on the enhancing particle.

FIG. 2a depicts a bead of this invention having a Raman enhancing particle with Raman markers attached thereto. The Raman enhancing particle is depicted within the bead and the bead has analyte receptors thereon.

FIG. 2b depicts a bead of this invention having a plurality of unlinked enhancing particles having Raman markers therein. The bead has analyte receptors on the outside.

FIG. 3a depicts a pair of enhancing particles of this invention linked together, and a Raman marker on the linker.

FIG. 3b depicts a pair of enhancing particles of this invention having a Raman marker associated with a particle.

FIG. 4a depicts an embodiment of this invention comprising a group of enhancing particles linked together. FIG. 4b depicts an embodiment of this invention as in FIG. 4a and having Raman markers on the enhancing particles.

FIG. 4c depicts an embodiment of this invention as in FIGS. 4a and 4b, and having analyte receptors attached to the enhancing particles.

FIG. 4d depicts an embodiment of this invention wherein a group of linked enhancing particles has Raman markers attached to the linkers.

FIG. 4e depicts an embodiment of this invention comprising linked enhancing particles having analyte receptors attached thereto and Raman markers attached to receptors.

FIG. 4f depicts an embodiment of this invention comprising linked enhancing particles having analyte receptors attached thereto, and having Raman markers attached to linkers, receptors or particles. Analytes are depicted associated with certain receptors.

FIG. 5a depicts an embodiment of this invention in which a bead has an enhancing particle therein having Raman markers. Analyte receptors on the surface of the bead have analyte molecules associated therewith.

FIG. 5b depicts an embodiment of this invention comprising a pair of linked enhancing particles and wherein Raman markers are associated with the linker. The bead has analyte receptors, and analyte molecules are depicted associated with analyte receptors.

FIG. 5c depicts an embodiment of this invention comprising a group of linked enhancing particles having Raman markers on the particles, the group of linked particles within a bead. Receptors are depicted on the surface of the bead and analyte molecules are shown associated with the receptors.

FIG. 6 depicts an embodiment of this invention wherein two beads have enhancing particle structures therein, each particle structure having different Raman markers.

FIG. 7 depicts a biochip having a plurality of Raman marked beads thereon in a random fashion, wherein each bead has a unique combination of Raman markers.

FIG. 8a depicts several beads or biomarkers of this invention, each having a unique combination of Raman markers and receptors specific for certain cells, tissues or pathogens.

FIG. 8b depicts a drawing of a section of a tissue having different cell, tissue and pathogens therein. Each type of cell, tissue and pathogen is labeled by biomarkers having receptors specific for the cell, tissue or pathogen type.

FIG. 9 depicts two beads of this invention having different combinations of Raman markers and different, synthesized chemical entities on the surfaces of the beads.

FIG. 10a depicts a Raman spectrum of mercaptopurine, a Raman marker used for making beads of this invention.

FIG. 10b depicts a Raman spectrum of DTP, a Raman marker used for making beads of this invention.

FIG. 10c depicts a Raman spectrum of dinitrophenol (DNP) cystine, a Raman marker used to make beads of this invention.

FIG. 11 a depicts a Raman spectrum of purine, a Raman marker used to make beads of this invention.

FIG. 11b depicts a Raman spectrum of mercaptoethylether (MEE), a Raman marker and a linker used to make beads of this invention.

FIG. 11c depicts a Raman spectrum of MEE and purine together.

FIG. 12 depicts a system for analyte detection of this invention.

### DETAILED DESCRIPTION OF THE INVENTION

Definitions

The following words and terms are used herein.

The term “analyte” as used herein means molecules, particles or other material whose presence and/or amount is to be determined. Examples of analytes include but are not limited to deoxyribonucleic acid (“DNA”), ribonucleic acid (“RNA”), amino acids, proteins, peptides, sugars, lipids, glycoproteins, cells, sub-cellular organelles, aggregations of cells, and other materials of biological interest.

The term “fractal” as used herein means a structure comprised of elements, and having a relationship between the scale of observation and the number of elements, i.e., scale-invariant. By way of illustration only, a continuous line is a 1-dimensional object. A plane is a two-dimensional object and a volume is a three-dimensional object. However, if a line has gaps therein, and is not a continuous line, the dimension is less than one. For example, if ½ of the line is missing, then the fractal dimension is ½. Similarly, if points on a plane are missing, the fractal dimension of the plane is between one and 2. If ½ of the points on the plane are missing, the fractal dimension is 1.5. Moreover, if ½ of the points of a solid are missing, the fractal dimension is 2.5. In scale invariant structures, the structure of objects appears to be similar, regardless of the size of the area observed. Thus, fractal structures are a type of ordered structures, as distinguished from random structures, which are not ordered.

The term “fractal associate” as used herein, means a structure of limited size, comprising at least about 100 individual particles associated together, and which demonstrates scale invariance within an area of observation limited on the lower bound by the size of the individual particles comprising the fractal associate and on the upper bound by the size of the fractal associate.

The term “fractal dimension” as used herein, means the exponent D of the following equation: N∝R^{D}, where R is the area of observation, N is the number of particles, and D is the fractal dimension. Thus in a non-fractal solid, if the radius of observation increases by 2-fold, the number of particles observed within the volume increases by 2³. However, in a corresponding fractal, if the radius of observation increases by 2-fold, the number of particles observed increases by less than 2³.

The term “fractal particle associates” as used herein means a large number of particles arranged so that the number of particles per unit volume (the dependent variable) or per surface unit changes non-linearly with the scale of observation (the independent variable).

The term “label” as used herein means a moiety having a physicochemical characteristic distinct from that of other moieties that permit determination of the presence and/or amount of an analyte of which the label is a part. Examples of labels include but are not limited to fluorescence, spin-resonance, radioactive moieties. Also known as reporter group.

The term “linker” as used herein means an atom, molecule, moiety or molecular complex having two or more chemical groups capable of binding to a surface and permitting the attachment of particles together to form groups of particles. The simplest linker connects two particles. A branched linker may link together larger numbers of particles. The term linkers includes those entities that are tri-functional, tetra-functional, or have even larger numbers of functional groups that can be used to link particles together.

The term “ordered structures” as used herein means structures that are non-random.

The term “particle structures” as used herein means a group of individual particles that are associated with each other in such a fashion as to permit enhancement of electric fields in response to incident electromagnetic radiation. Examples of particles include metals, metal-coated polymers and fullerenes. Also included in the meaning of the term “particle structures” are films or composites comprising particles on a dielectric surface or imbedded in a dielectric material.

The term “Raman array reader” as used herein means a device having a light source and a light detector.

The term “Raman marker” as used herein means a molecule or moiety having a characteristic Raman spectral feature or an identifiable pattern of spectral features that permits identification of that marker either by itself, or in a mixture of other Raman markers. Even in situations in which the same Raman spectral features are common to one or more markers, if ratios of intensities of the spectral features permit unique identification of the marker or groups of markers, then the moiety is considered a Raman marker for purposes of this application.

The term “Raman signal” as used herein means a Raman spectrum or portion of Raman spectrum.

The term “Raman spectral feature” as used herein means a value obtained as a result of analysis of a Raman spectrum produced for an analyte under conditions of detection. Raman spectral features include, but are not limited to, Raman band frequency, Raman band intensity, Raman band width, a ratio of band widths, a ratio of band intensities, and/or combinations the above.

The term “Raman spectroscopy” as used herein means a method for determining the relationship between intensity of scattered electromagnetic radiation as a function of the frequency of that electromagnetic radiation.

The term “Raman spectrum” as used herein means the relationship between the intensity of scattered electromagnetic radiation as a function of the frequency of that radiation.

The term “random structures” as used herein means structures that are neither ordered nor fractal. Random structures appear uniform regardless of the point and scale of observation, wherein the scale of observation encompasses at least a few particles.

The term “receptor” as used herein means a moiety that can bind to or can retain an analyte or other molecule of interest.

The term “resonance” as used herein means an interaction with either incident, scattered and/or emitted electromagnetic radiation and a surface having electrons that can be excited by the electromagnetic radiation and increase the strength of the electric field of the electromagnetic radiation.

The term “resonance domain” as used herein means an area within or in proximity to a particle structure in which an increase in the electric field of incident electromagnetic radiation occurs.

The term “reporter group” as used herein means a label.

The term “scaling diameter” as used herein means a relationship between particles in a nested structure, wherein there is a ratio (scaling ratio) of particle diameters that is the same, regardless of the size of the particles.

The term “surface enhanced Raman spectroscopy” (“SERS”) as used herein means an application of Raman spectroscopy in which intensity of Raman scattering is enhanced in the presence of an enhancing surface.

The term “surface enhanced resonance Raman spectroscopy” (“SERRS”) as used herein means an application of Raman spectroscopy in which Raman signals of an analyte are enhanced in the presence of an enhancing surface (see SERS) and when an absorption band of the analyte overlaps with the wavelength of incident electromagnetic radiation.

Embodiments of the Invention

The methods and compositions of this invention represent improvements over the existing methods for marking beads for a variety of uses including analyte detection and combinatorial syntheses. In particular, the compositions and methods can be desirable for use in conjunction with infrared spectroscopy, fluorescence spectroscopy, surface plasmon resonance, mass spectroscopy or any other method utilizing excitation of an analyte by electromagnetic radiation and the emission of a signal characteristic of that analyte.

Certain embodiments of this invention are based upon Surface Enhanced Raman Spectroscopy (“SERS”), and Surface Enhanced Resonance Raman Spectroscopy (“SERRS”). This invention includes methods for manufacturing Raman active structures having specific markers and/or receptor molecules attached to those structures. The invention also includes methods for detecting markers using Raman spectroscopy, and arrays and test kits embodying Raman spectroscopic methods for identifying specific beads used to detect analytes.

The structures that are desirable for use according to the methods of this invention include structures of small particles in structures, herein termed particle structures, nanoparticle structures, and/or enhancing particles, which includes as a subset, fractal associates. Particle structures can be characterized by having physical and chemical structures that enable oscillations of electrons to be in resonance with incident and outgoing electromagnetic radiation.

In certain embodiments of this invention, markers for recognition of beads are moieties providing a Raman signal (or other optical signals known to those skillful in the art as SERS, SERRS, SEHRS, SEHRRS, as well as SEIRA) that are in close proximity to enhancement surface of aggregated, aggregated and linked, or linked nanoparticles, rather than a single nanoparticle disclosed in a prior art. WO 01/25758 A1 “Surface Enhanced Spectroscopy-Active Composite Nanoparticles”, Michael J. Natan, incorporated herein fully by reference. The advantage of using particle pairs, particle aggregates, linked aggregates or linked complexes of nanoparticles include broader range of wavelengths that can be enhanced, and the magnitude of the enhancement can be substantially greater than for a single particle.

To obtain an optical response under enhancing conditions, the wavelength of incident electromagnetic radiation can overlap with an absorbance band of plasmon wave of metal particles producing the enhancement. Such absorbance band for a single nanoparticle is rather narrow, whereas it is much broader for Assembled Particle Structures. Typically lasers are used as the source of incident electromagnetic radiation and these light sources are available only for a limited set of wavelengths. Tunable lasers covering broad range of wavelengths are expensive, have poor stability over time and generally require a trained professional to maintain such a light source, which makes such lasers impractical as a part of reliable and affordable instrumentation for recognition of beads at present. Thus, one is limited with only a few options in the choice of a light source when uses single nanoparticle-based markers.

According to one theory of resonance enhancement, when a wavelength of incident electromagnetic radiation overlaps with an absorbance band of the marker moiety, the magnitude of a signal can be increased. This enhancement can result, in some cases, in 10⁸-fold increase in Raman signal. This enhancement allows one to substantially decrease the power of light source without losing the intensity of the signal, i.e., one can use less expensive lasers. The selection of laser sources compatible not only with the goal of recognition of a bead (i.e., having wavelength overlapping both with the absorbance band of the moiety providing a signal and with the narrow surface plasmon band), but also with inducing a fluorescence response from analyte bound to a receptor associated with the beads is a challenging task. Particle structures of this invention can allow the use of a variety of lasers. This allows one to easily select laser sources compatible not only with the goal of recognition of a bead, but also with inducing a fluorescence response from analyte bound to a receptor associated with the beads of this invention.

As compared to an individual nanoparticle, a plurality of particles (e.g., two or more) can provide higher enhancement of optical signals of moieties in close proximity thereof. The enhancement of a signal derived from a single particle is not as strong as that derived using a plurality of particles because single particle does not provide a phenomenon known as a hot spot, that is an enhancement of electromagnetic field in some areas in a proximity to a particle structure. Such hot-spots may be produced more frequently in structures comprising a plurality of particles. When a moiety is present in such a hot spot area, its Raman signal is so strong that the presence of a single molecule may be sufficient for recognition of a bead. Such high signals from individual nanoparticles are not achieved cannot be achieved using prior art particles.

I. Manufacture of Particle Structures

The Raman active structures desirable for use according to this invention can include any structure in which Raman signals can be amplified. The following discussion regarding metal fractal structures is not intended to be limiting to the scope of the invention, but is for purposes of illustration only.

A. Manufacture of Metal Particles

To make metal particles for nanoscale arrays of receptors according to some embodiments of this invention, we can generally use methods known in the art (Tarcha et al., U.S. Pat. No. 5,567,628, incorporated herein fully by reference). Metal colloids can be composed of noble metals, specifically, elemental gold or silver, copper, platinum, palladium and other metals known to provide surface enhancement. In general, to make a metal colloid, a dilute solution containing the metal salt is chemically reacted with a reducing agent. Reducing agents can include ascorbate, citrate, borohydride, hydrogen gas, and the like. Chemical reduction of the metal salt can produce elemental metal in solution, which combine to form a colloidal solution containing metal particles that are relatively spherical in shape. By way of example, particles can be made using one or more methods disclosed in U.S. patent application Ser. No. 09/925,189, herein incorporated fully by reference.

Example 1: Manufacture of Gold Colloid and Fractal Structures In one embodiment of this invention, a solution of gold nuclei is made by preparing a 0.01% solution of NaAuCl₄ in water under vigorous stirring. One milliliter (“ml”) of a solution of 1% sodium citrate is added. After 1 minute of mixing, 1 ml of a solution containing 0.075% NaBH₄ and 1% sodium citrate is added under vigorous stirring. The reaction is permitted to proceed for 5 minutes to prepare the gold nuclei having an average diameter of about 2 nm). The solution containing the gold nuclei can be refrigerated at 4° C. until needed. This solution can be used as is, or can be used to produce particles of larger size (e.g., up to about 50 nm diameter), by rapidly adding 30 μl of the solution containing gold nuclei and 0.4 ml of a 1% sodium citrate solution to the solution of 1% HAuCl₄3H₂O diluted in 100 ml H₂O, under vigorous stirring. The mixture is boiled for 15 minutes and is then cooled to room temperature. During cooling, the particles in the solution can form fractal structures. The resulting colloid and/or fractal particle structures can be stored in a dark bottle.

Deposition of enhancing particles on dielectric surfaces including glass can generate films that can enhance electromagnetic signals. Such films can be as thin as about 10 nm. In particular, the distribution of electric field enhancement on the surface of such a film can be uneven. Such enhancing areas are resonance domains. Such areas can be particular useful for positioning receptors for analyte binding and detection. For films or particle structures embedded in dielectric materials, one way to manufacture enhancing structures is to treat the surface until “percolation points” appear. Methods for measuring sheet resistance and bulk resistance are well known in the art.

### EXAMPLE 2

### Manufacture of Metal Particles and Fractal Structures Using Laser Ablation

In addition to liquid phase synthesis described above, laser ablation is used to make metal particles. A piece of metal foil is placed in a chamber containing a low concentration of a noble gas such as helium, neon, argon, xenon, or krypton. Exposure to the foil to laser light or other heat source causes evaporation of the metal atoms, which, in suspension in the chamber, can spontaneously aggregate to form fractal or other particle structures as a result of random diffusion. These methods are well known in the art.

B. Manufacture of Films Containing Particles

To manufacture substrates containing metal colloidal particles of one embodiment of this invention, the colloidal metal particles can be deposited onto quartz slides as described herein. Other films can be made that incorporate random structures or non-fractal ordered structures in similar fashions. Additionally, films comprising beads having Raman markers are included in this invention.

### EXAMPLE 3

### Manufacture of Quartz Slides Containing Gold Fractal Structures

Quartz slides (2.5 cm×0.8 cm×0.1 cm) are cleaned in a mixture of HCl:HNO₃ (3:1) for several hours. The slides are then rinsed with deionized H₂O (Millipore Corporation) to a resistance of about 18 MΩ and then with CH₃OH. Slides are then immersed for 18 hours in a solution of aminopropyltrimethoxysilane diluted 1:5 in CH₃OH. The slides are then rinsed extensively with CH₃OH (spectrophotometric grade) and deionized H₂O prior to immersion into colloidal gold solution described above. The slides are then immersed in the gold colloid solution above. During this time, the gold colloid particles can deposit and can become attached to the surface of the quartz slide. After 24 hours, colloid derivatization is complete. Once attached, the binding of colloidal gold nanocomposites to the quartz surfaces is strong and is essentially irreversible. During the procedure, ultraviolet and/or visual light absorbance spectra of such derivatized slides are used to assess the quality and reproducibility of the derivatization procedure. The manufacturing process is monitored using electron microscopy to assess the density of the colloidal coating, the distribution of gold colloid particles on the surface, and the size of the gold colloid particles.

C. Aggregation of Particles to Form Particle Structures

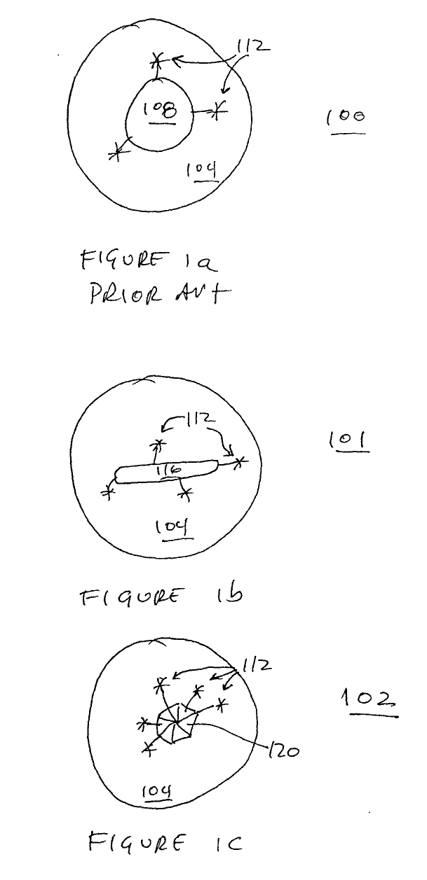

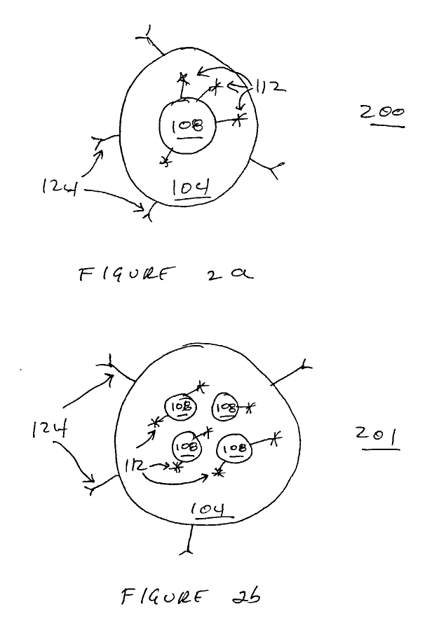

According to other embodiments of this invention, several methods can be used to form particle structures. It is known that metal colloids can be deposited onto surfaces, and when aggregated can form fractal structures having a fractal dimension of about 1.8. Safonov et al., Spectral Dependence of Selective Photomodification in Fractal Aggregates of Colloidal Particles, Physical Review Letters 80(5):1102-1105 (1998) incorporated herein fully by reference. FIG. 1 depicts a particle structure suitable for use with the methods of this invention. The particles are arranged in a scale-invariant fashion, which promotes the formation of resonance domains upon illumination by laser light.

In addition to fractal structures, ordered non-fractal structures and random structures can be generated. These different types of structures can have desirable properties for enhancing signals associated with detection of analytes using electromagnetic radiation. To make ordered non-fractal structures, one can use, for example, chemical linkers having different lengths sequentially as described in more detail below. In addition, using linkers of the same size, one can generate ordered structures, which can be useful for certain applications.

In certain embodiments of this invention, particles can be attached together to form structures having resonance properties. In general, it can be desirable to have the particles being spheres, ellipsoids, or rods. For ellipsoidal particles, it can be desirable for the particles to have a long axis (x), another axis (y) and a third axis (z). In general, it can be desirable to have x be from about 0.05 to about 1 times the wavelength (λ) of the incident electromagnetic radiation to be used. For rods, it can be desirable for x to be less than about 4λ, alternatively, less than about 3λ, alternatively less than about 2λ, in other embodiments, less than about 1λ, and in yet other embodiments, less than about ½λ. The ends of the rods can be either flat, tapered, oblong, or have other shape that can promote resonance.

For two particle structures, it can be desirable for the particle pair to have an x dimension to be less than about 4λ, alternatively, less than about 3λ, alternatively less than about 2λ, in other embodiments, less than about 1λ, and in yet other embodiments, less than about ½λ.

For two-dimensional structures, pairs of particles, rods, rods plus particles together can be used. The arrangement of these elements can be randomly distributed, or can have a distribution density that is dependent upon the scale of observation in a non-linear fashion.

In other embodiments, rods can be linked together end-to end to form long structures that can provide enhanced resonance properties.

For three-dimensional structures, one can use regular nested particles, or chemical arrays of particles, associated either by chemical linkers in a fractal structure or in ordered, nested arrays.

In yet other embodiments, of third-order structures, a suspension of particles can be desirable. In certain of these embodiments, the suspended particles can have dimensions in the range of about ½λ to about 1 millimeter (mm).

Using the strategies of this invention, are searcher or developer can satisfy many needs, including, but not limited to selecting the absorbance of electromagnetic radiation by particle elements, the nature of the surface selected, the number of resonance domains, the resonance properties, the wavelengths of electromagnetic radiation showing resonance enhancement, the porosity of the particle structures, and the overall structure of the particle structures, including, but not limited to the fractal dimensions of the structure(s).

1. Photoaggregation

Photoaggregation can be used to generate particle structures that have properties which can be desirable for use in Raman spectroscopy.

Irradiation of fractal metal nanocomposites by a laser pulse with an energy above a certain threshold leads to selective photomodification, a process that can result in the formation of “dichroic holes” in the absorption spectrum near the laser wavelength (Safonov et al., Physical Review Letters 80(5):1102-1105 (1998), incorporated herein fully by reference). Selective photomodification of the geometrical structure can be observed for both silver and gold colloids, polymers doped with metal aggregates, and films produced by laser evaporation of metal targets.

One theory for the formation of selective photomodification is that the localization of optical excitations in fractal structures are prevalent in random nanocomposites. According to this theory, the localization of selective photomodification in fractals can arise because of the scale-invariant distribution of highly polarizable particles (monomers). As a result, small groups of particles having different local configurations can interact with the incident light independently of one another, and can resonate at different frequencies, generating different domains, called herein “optical modes.” According to the same theory, optical modes formed by the interactions between monomers in fractal are localized in domains that can be smaller than the optical wavelength of the incident light and smaller than the size of the clusters of particles in the colloid. The frequencies of the optical modes can span a spectral range broader than the absorption bandwidth of the monomers associated with plasmon resonance at the surface. However, other theories may account for the effects of photomodification of fractal structures, and this invention is not limited to any particular theory for operability.

Photomodification of silver fractal aggregates can occur within domains as small as about 24×24×48 nm³ (Safonov et al., Physical Review Letters 80(5:1102-1105 (1998), incorporated herein fully by reference). The energy absorbed by the fractal medium can be localized in a progressively smaller number of monomers as the laser wavelength is increased. As the energy absorbed into the resonant domains increases, the temperature at those locations can increase. At a power of 11 mj/cm², light having a wavelength of 550 nm can produce a temperature of about 600 K (Safonov et al., Physical Review Letters 80(5): 1102-1105 (1998), incorporated herein fully by reference). At this temperature, which is about one-half the melting temperature of silver, sintering of the colloids can occur (Safonov et al., Id.) incorporated herein fully by reference), thereby forming stable fractal nanocomposites.

As used in this invention, photoaggregation can be accomplished by exposing a metal colloid on a surface to pulses of incident light having a wavelengths in the range of about 400 nm to about 2000 nm. In alternative embodiments, the wavelength can be in the range of about 450 nm to about 1079 nm. The intensity of the incident light can be in the range of about 5 mJ/cm² to about 20 mj/cm². In an alternative embodiment, the incident light can have a wavelength of 1079 nm at an intensity of 11 mJ/cm².

Fractal aggregates that are especially useful for the present invention can be made from metal particles having dimensions in the range of about 10 m to about 100 nm in diameter, and in alternative embodiments, about 50 nm in diameter. A typical fractal structure of this invention is composed of up to about 1000 particles, and an area of the aggregate typically used for large-scale arrays can have a size of about 100 μm×100 μm.

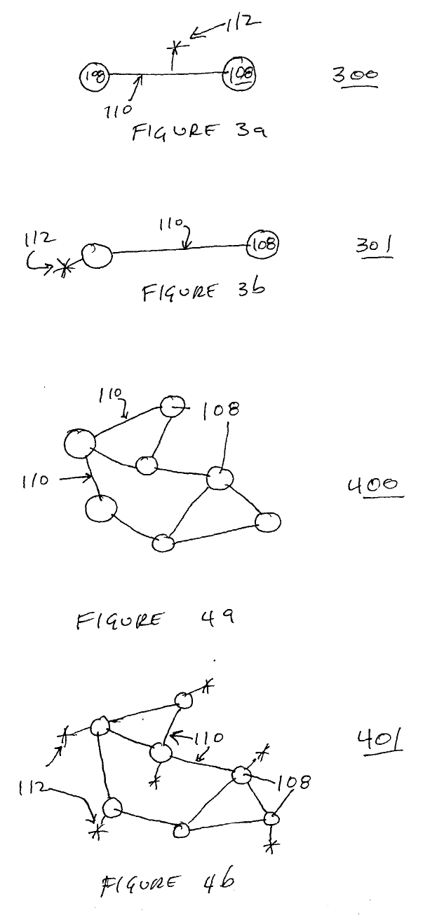

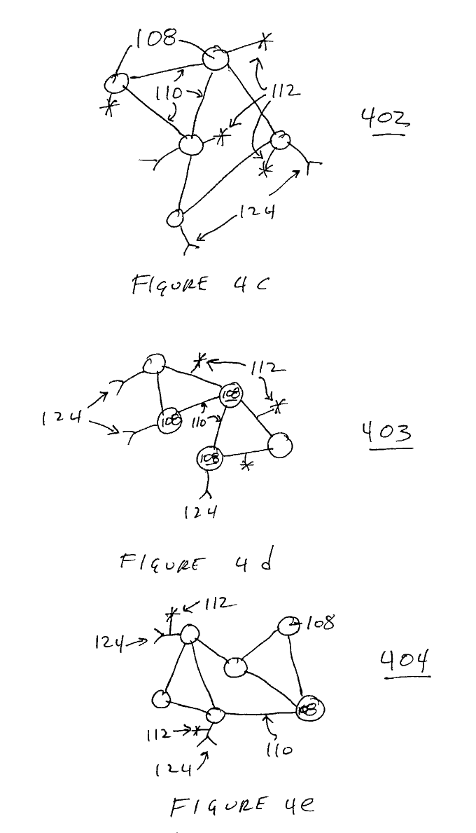

FIG. 2 depicts a particle structure that have been photoaggregated and that are suitable for use with the methods of this invention. Local areas of fusion of the metal particles can be observed (circles).

2. Chemically Directed Synthesis of Particle Structures

In certain embodiments of this invention, particle structures can be made using chemical methods. First, metal particles can be either made according to methods described above, or alternatively can be purchased from commercial suppliers (NanoGram Inc., Fremont, Calif.). Second, the particles can be joined together to form first-order structures, for example, pairs of particles. Then, the first-order structures can be joined together to form second-order structures, for example, pairs of particle pairs. Finally, third-order fractal structures can be made by joining second-order structures together.

In alternative embodiments of this invention, the formation of a fractal array of metal particles can be carried out using chemical methods. Once metal colloid particles have been manufactured, each particle can be attached to a linker molecule via a thiol or other type of suitable chemical bond. The linker molecules then can be attached to one another to link adjacent colloid particles together. The distance between the particles is a function of the total lengths of the linker molecules. It can be desired to select a stoichiometric ratio of particles to linker molecules. If too few linker molecules are used, then the array of particles will be too loose or may not form at all. Conversely, if the ratio of linker molecules to particles is too high, the array may become too tight, and may even tend to form crystalline structures, which are not random, and therefore will not tend to promote surface enhanced Raman scattering.

In general, it can be desirable to perform the linking procedure sequentially, wherein the first step comprises adding linker molecules to individual particles under conditions that do not permit cross-linking of particles together. By way of example only, such a linker can comprise an oligonucleotide having a reactive group at one end only. During this first step, the reactive end of the oligonucleotide can bind with a metal particle, thereby forming a first particle-linker species, and having a free end of the linker. The ratio of linker molecules to particles can be selected, depending on the number of linker molecules are to be attached to the particle. A second linker can be attached to another group of particles in a different reaction chamber, thereby resulting in a second linker-particle species, again with the linker having a free end.

After those reactions have progressed, the different linker-particle species can be mixed together and the linkers can attach together to form “particle pairs” joined by the linker molecules.

Nanoparticles of a number of coinage metals, such as Au, Ag, Cu, Pt, display surface plasmon resonance in visible or near infrared spectral range. These particles are most suitable for enhancement of Raman and other optical responses from moieties that are in a close proximity to the surface of such particles. Colloidal solutions of gold or silver particles can be prepared as described in Examples (see earlier applications), using other protocols known in the art of colloidal chemistry. Alternatively, nanoparticles of various kinds can be prepared by laser ablation.

Manufacturing of linked nanoparticles can be performed by linking particles together using linker molecules or molecular complexes (linkers). These linkers can be rod-shape molecules having two moieties capable of tight, essentially irreversible binding to a metal surface. Typically, these moieties are thiol groups, however other chemistries can be used. Another known in the art way of linking of two kinds of nanoparticles, each derivatized with either of two non-complimentary oligonucleotides having an alkyl-thiol moiety, is achieved by adding a DNA linker having complementary oligonucleotides to both sequences at its ends. Thus, it is known to those skillful in the art that linear link of two particles can be obtained by adding a rod-shape linker capable of attachment of two particles together.

Linking of more than two particles can be achieved by using quatanary linkers of this invention. These quatanary linkers are molecules that have more than two moieties capable of binding to a metal surface. The examples of quatanary linkers include but are not limited to pentaerythritol tetrakis(2-mercaptoacetate) and pentaerythritol tetrakis(3-mercaptopropionate). One can also link nanoparticles to form even more complex linear-spatial complexes using reagents composed of two or more quaternary thiol moieties linked together by using a chemical linker connecting two thiols.

Another way of this invention to form linked nanoparticle complexes of this invention is as follows. Addition of salt, for example NaCl, in a concentration sufficient to induce aggregation of nanoparticles in colloidal solution can produce aggregates of nanoparticles, including fractal aggregates. Those skillful in the art are able to generate essentially fractal aggregates of nanoparticles. These aggregates are not stable because interactions between particles in such aggregates are week. By adding a linker to such aggregates, one achieves formation of In general, to achieve formation of linked nanoparticles, linkers can be added to a colloidal solution of nanoparticles. Those skillful in the art are able to identify a concentration of linkers that is sufficient to produce linked particles in sufficient amounts. It can be desirable to carefully perform several experiments as described in Example 2 to identify preferable concentration of linker to be used.

### EXAMPLE 4

### Manufacturing of Colloidal Fractal Aggregates Linked with Mercaptoethylether

A colloidal solution of silver nanoparticles is prepared using sodium citrate reduction as described earlier. Upon addition of NaCl to final concentration 200 mM to the solution, formation of aggregated nanoparticles occur within several minutes. Addition of mercaptoethylether to final concentration 1 mM results in complete coverage of the surface of the particles and sufficient linking of particles to produce stable aggregates. The completeness of coverage and according degree of linking can be varied by decreasing the concentration of mercaptoethylether

### EXAMPLE 5

### Procedure for Controlling the Degree of Surface Coverage in Colloidal Fractal Aggregates Linked with Mercaptoethylether

When rod-shape linkers bind to nanoparticles, each binding moiety of such a linker can either (a) bind to one particle, or (b) to two particles producing linking between particles, or (c) such linker can bind only via one of its binding moieties, whereas its second moiety remains free. The case (b) result in strengthening of nanoparticles in an aggregate, whereas the cases (a) and (c) result in reducing of free metal surface. For example, at concentration of mercaptoethylether 1 mM (see Example above), complete covering of the metal surface occurs resulting in the loss of ability of other compounds capable of binding to the surface to attach (the surface is thus passivated by the linker). The degree of covering is thus important characteristic to control the process of linking and concomitant passivation. This degree can be obtained in the following procedure: One prepares thiol-containing gold surface by overnight incubation of Deposition Controller Quarts Slides in 1 mM solution of mercaptoethylether. The controllers are washed extensively with water, isopropyl alcohol, water and 2 mM TCEP/HCl (PIERS) water solution, and than again with water to remove unbound and disulfide-linked mercaptoethylether molecules.

Aggregates of particles with varying degree of linking/passivation are prepared as described above using concentrations of mercaptoethylether varying from 1 mM to 10 nM. An aliquots of 250 microliter are applied onto mercaptoethylether-activated controller and incubated for 40 min at room temperature. Upon washing the slides with deionized, triply distilled water, isopropyl alcohol and again water, one measures the Raman Spectrum of the slide (785 nm excitation, 40 s integration time, 100 mWpower at the sample). Characteristic Raman bands of mercaptoethylether can be clearly seen. These bands are more intensive when higher concentration of mercaptoethylether is used for linking. If mercaptopurine (1 mM final) is added to such a surface, depending on the degree of linking/coverage, one can see either appearing of characteristic bands of mercaptopurine (low coverage/fewer links between particles), which is typical for 1-10 microM of mercaptoethylether or lower, or a very small signal or no of mercaptopurine (almost complete or complete coverage/many links between particles).

In certain cases, in which hydrophobic linkers are used to link particles to form enhancing structures, it can be desirable to use surfactants, which can improve the physical stability of the structures, thereby increasing the shelf life and increasing the robustness of the structures when they are used under harsh chemical conditions.

After the pairs of particles are formed, additional linkers can be attached to the particle pairs, and the process can be repeated to form “pairs of particle pairs.” Subsequently, the process can be repeated until 3 or more orders of particle structures are formed. Under these conditions, one can manufacture structures having any desired porosity. In general, the size of the nanoscale structures should have average dimensions in the range of about 20 nm to about 500 nm. In alternative embodiments, the dimensions can be in the range of about 50 nm to about 300 nm, and in other embodiments, in the range of about 100 to about 200 nm, and in yet other embodiments, about 150 nm.

In other embodiments of this invention, the linking can be carried out using an aryl dithiol or di-isonitrile molecules or any other moiety that can be used to attach the linker to the metal particle. In certain embodiments, one can use dithioldiethylether, although it can be appreciated that other types of dithiolethers can be used. Alternatively, one can use one or more of a variety of dithiolalkyl linkers, such as by way of example, HS-(CH₂)n-SH, wherein n is an integer of from 1 to about 20, alternatively from about 2 to about 10, or in yet other embodiments, about 2. To manufacture groups of particles having stronger 3-dimensional structure, one can use linkers having more than two functional groups. By way of example, pentaerythritol tetrakis(2-mercaptoacetate), pentaerythritol tetrakis (3-mercaptopropionate) and like molecules can be used. These types of linkers can provide improved mechanical strength to the group of enhancing particles, so that selection of linked groups can be more easily accomplished.

To purify selected types of particle clusters, after synthesis of linked groups, the mixture can be applied to a size-exclusion chromatography column and the different types of clusters separated using standard methods. In this fashion, one can select groups of linked particles having similar size, thereby permitting a greater degree of control over the particles used to identify beads.

In general, the ratio of length for each subsequent pairs of linkers can be in the range of about 2 to about 20. Alternatively, the ratios of lengths of subsequent pairs of linkers can be in the range of about 3 to about 10, and in other embodiments, about 5. In certain other embodiments, the ratio of linker lengths in successive orders can be non-constant, thus resulting in the manufacture of an ordered, non-fractal structure.

For example, for a three-order manufacturing process, it can be desirable for the ration of L1:L2:L3 to be in the range of about 1:2:4. Alternatively, the ratio can be about 1:5:25, and in yet other embodiments, the ratio can be about 1:20:400. In other embodiments, the ratio between L1 and L2 and from L2 to L3 need not be the same. Thus, in certain embodiments the ration of L1:L2:L3 can be 1:3:20, or alternatively, 1:20:40.

3. Manufacture of Suspensions of Fractal Particle Associates

In certain other embodiments of this invention, suspensions of fractal particle associates (fractal associates) can be used, for example, to provide a structure in solution that can bind or retain analytes for detection using methods of this invention. The size of fractal particle associates can be in the range of from hundreds of nanometers to mm dimensions. The fractal associates can comprise a number of particles arranged by means of chemical linkers. The number of particles per fractal associate can be as few as about 100 particles, or alternatively, thousands can be used to form a fractal associate. By increasing the number of particles in a fractal associate, the increase in the void size increases by a greater proportion.

II. Selection of Raman Markers

To take full advantage of the numbers of possible identifying moieties for beads of this invention, one can use a relatively small number of different Raman markers in different combinations with each other. In general, Raman markers that are suitable for this invention include any molecule or moiety having an identifiable Raman signal that has at least one Raman spectral feature different from all of the other potential Raman markers, and wherein the Raman marker can bind to enhancing particles and/or linkers and/or analyte receptors. It can be appreciated that is not necessary that all of the Raman spectral features of different markers be different from each other. Rather, it is only necessary to be able to distinguish each marker from the other. For example two markers may have identical Raman spectral features present. However, if relative intensities of two different features differ between the two markers, then it is possible, through deconvolution methods known in the art, to detect the presence of the two markers present in the same sample.

One aim of this invention is to provide a relatively large number of distinguishable markers and combinations of markers to permit the detection and identification of a large number of different beads. The binary theorem provides a convenient way to determine the number of markers needed to uniquely identify any desired number of different beads. For example, it can be readily appreciated that if one uses two different markers, a total of 3 identifiable marker combinations is available. For example if the two markers and “a” and “b”, then one can use “a” alone, “b” alone, or “a+b”, thereby providing 3 different combinations of markers. Similarly, if one uses “a,” “b” and “c” markers, one can use “a”, “b”, “c”, “a+b”, “a+c,” and “a+b+c” for a total of seven combinations. It can be readily appreciated that the total number of combinations of n different markers, taken 1 to n at a time is represented by the expression: 2ⁿ−1. Thus, by using 10 different markers, one can identify 1023 different beads, and by using 20 different markers, one can identify over 4 million different beads. Thus, the total number of markers can be limited by the total number of identifiable beads desired. For many applications, it may be necessary to identify 10,000 of so different beads, in which case 12 markers is sufficient. Similarly to identify 1,000,000 different beads one need only 18 different markers.

The types of marker moieties is not limited, but rather can be expansive. However, for convenience of manufacture, it can be desirable to use markers selected from thiol-, amino and/or aromatic moieties. For example, it can be desirable to use markers including mercaptopurine, dithiodipyridine, dinitrophenol cystine, rhodamine 6-G, purine, mercaptoethanol, mercaptoethylamine, dithioldiether, mercaptoethyl ether, ethane dithiol, mercaptosuccinic acid, pentaerythritol tetrakis(2-mercaptoacetate) and pentaerythritol tetrakis (3-mercaptopropionate). When using aromatic markers, it can be desirable to use an aromatic moiety comprising a heteroatom, such as nitrogen to provide sufficiently characteristic Raman signals.

III. Design and Manufacture of Beads Having Raman Markers

Once enhancing particles are manufactured, they can be introduced into beads. In general, beads can be made of any material that is capable of supporting the desired receptor, and does not have its own Raman signal that would interfere with the signals produced by the Raman markers. For convenience only, as used herein, the material from which a bead may be made is called a “matrix.” It can be appreciated that crystalline structures, polymers, as well as amorphous structures may also provide satisfactory matrices for the bead. Additionally, when beads are used for fluorescent analyte detection, it is desirable for the bead material to not produce a fluorescent signal in the wavelength band of the fluorescently labeled analyte.

Beads can be of any of a variety of convenient sizes, and for many uses can be in the range of about 500 nm to about 5 μm in diameter. For use with enhancing structures, beads can have a size in the range of about 2 to about 10 times the diameter of the enhancing particle structure. Raman enhancing structures can have sizes in the range of about 20 nm to about 100 nm, although in some cases, larger structures maybe suitable. The size of the bead/enhancing structure combination can depend on the wavelengths of electromagnetic radiation used to produce the Raman signal of the marker. Thus, it can be desirable to have enhancing particle structures having sizes of about 12 the wavelength of the electromagnetic radiation.

Beads may conveniently made having a porous structure with interstices present. Such porous beads can permit the introduction of enhancing particle structures to the interior of the beads. Many methods are available for manufacture of beads having enhancing particle structures therein. For example, one may prepare beads of a swellable polymer material, soak the beads in a solvent, permit the beads to swell as solvent is taken into the interstices. Then, a solution containing enhancing particle structures having Raman markers thereon can be introduced. Enhancing particle structures can diffuse into the interior of the bead. Then, the solvent can be removed, for example, by evaporation or solvent exchange, and certain of the enhancing particle structures can be trapped within the bead. Then, residual particle structures may be washed from the exterior surfaces of the beads, thereby resulting in a bead having trapped enhancing particle structures therein.

Subsequently, the beads may have specific receptors attached thereto. Many methods are available for attaching receptors to surfaces. It may be desirable that the conditions and reagents used should desirably not interfere either with the specificity of binding of the receptor to its corresponding ligand, or adversely alter the configuration of the enhancing structures within the beads.

FIG. 1a depicts a prior art bead 100 having a bead matrix 104, a single, spherical enhancing particle 108, and having Raman markers 112 attached thereto.

FIG. 1b depicts an embodiment 101 of this invention, having bead matrix 104, rod-shaped enhancing particle 116 having Raman markers 112 attached thereto, either on the end or on the mid-portion of rod-shaped particle 116.

FIG. 1c depicts an embodiment 102 of this invention, having bead matrix 104, and crystalline enhancing structure 120 therein, having Raman markers 112 thereon, attached to vertices, apex, or facet of crystalline structure 120.

IV. Manufacture of Receptor-Derivatized Beads

Once the particle structures of metal particles have been manufactured, receptors can then be attached, thereby forming receptor-derivatized structures that are useful for spectroscopic detection and quantification of analytes.

A. Selection of Receptor

The receptor chosen to be attached to particle structures of this invention will depend on binding properties of the desired analyte. For example, to detect and quantify nucleic acid sequences, it can be desirable to use oligonucleotide receptors. Oligonucleotide receptors can hybridize to analyte nucleotide sequences, thereby producing a bound ligand. Alternatively, if desired, one can use an antibody directed against a nucleotide sequence to bind the nucleic acid. In other embodiments, DNA binding proteins can be used. For example, to detect certain promoter regions of genes, specific promoter-binding proteins can be used as receptors. Moreover, or peptide nucleic acids can be used to bind native nucleic acids.

Similarly, to detect protein analytes, antibodies and other, specific protein binding molecules can be used. Once the type of analyte is chosen, the specific receptor molecule and the conditions for its attachment to the fractal array can be determined. Additionally, antibodies directed against low molecular weight analytes can be attached to a substrate.

By way of example, the nucleic acid receptors can advantageously used in a large scale matrix array to measure a large number of analyte sequences simultaneously.

B. Manufacture of Beads Having Raman Markers and Receptors

FIG. 2a depicts an embodiment 200 of this invention having bead matrix 104, enhancing particle 108 therein having Raman markers 112 thereon, and having receptors 124 attached to the surface of the bead.

FIG. 2b depicts an embodiment 201 of this invention having bead matrix 104 a plurality of enhancing particles 108 therein having Raman markers 112 thereon, and having receptors 124 attached to the surface of the bead.

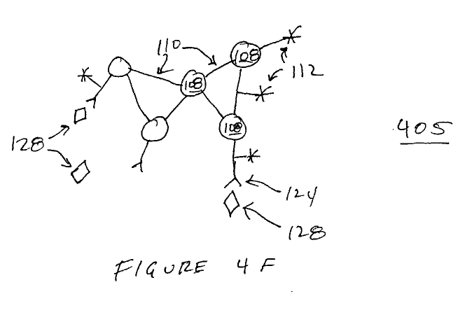

FIG. 3a depicts an embodiment 300 of this invention comprising a pair of particles 108 linked together with linker 110. Raman marker 112 is depicted attached to or part of linker 110. FIG. 3b depicts an alternative embodiment 301 of this invention, in which a pair of particles 108 are attached together with linker 110, and Raman marker 112 is attached to the particles 108.

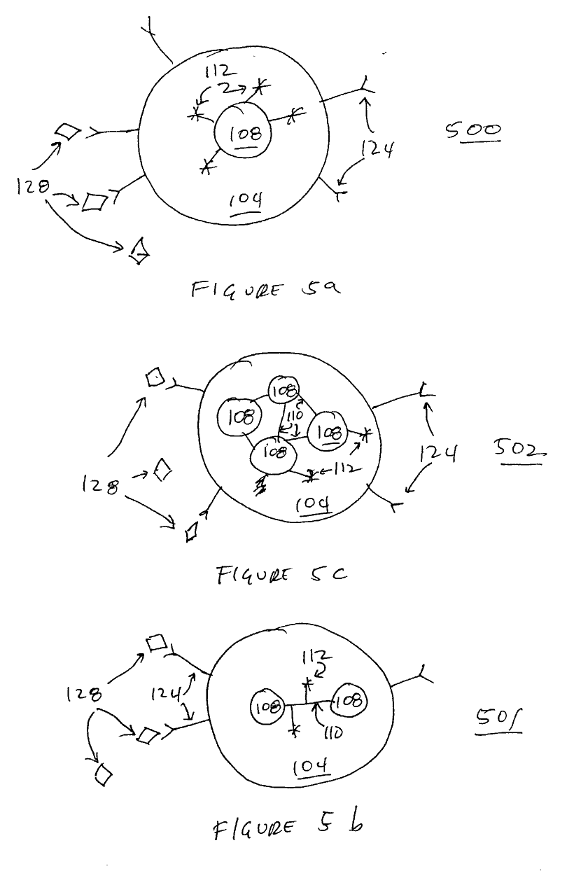

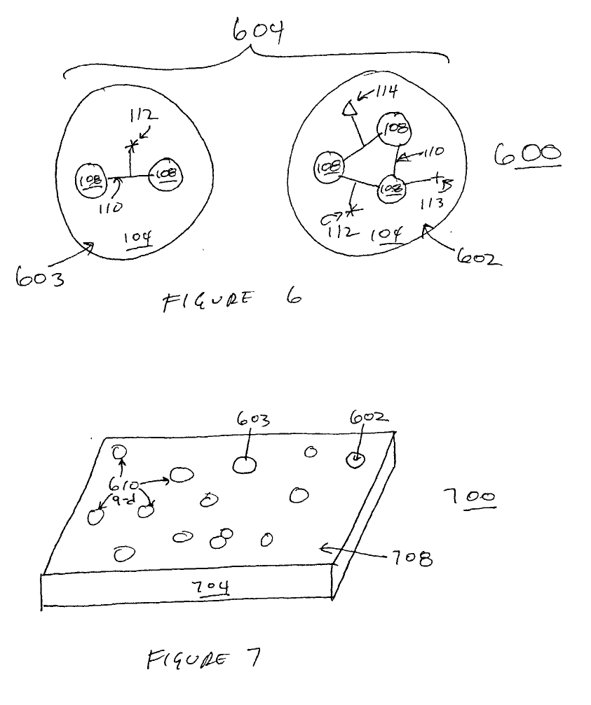

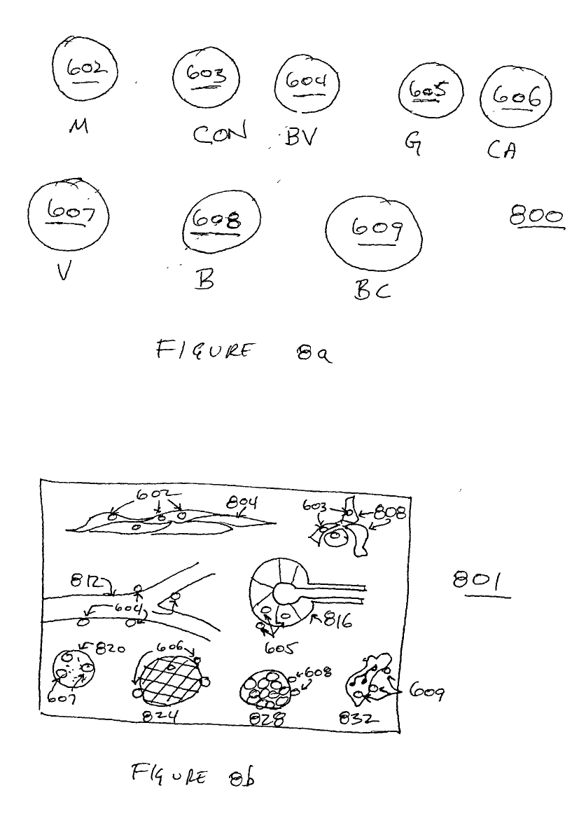

FIG. 4a depicts an alternative embodiment 400 of this invention, in which a plurality of particles 108 are linked together by a plurality of linkers 110.

FIG. 4b depicts an embodiment 401 of this invention as in FIG. 4a with the addition of Raman markers 112 attached to particles 108.

FIG. 4c depicts an alternative embodiment 402 of this invention. Particles 108 are attached together by linkers 110 and Raman markers 112 and receptors 124 are attached or associated with particles 108.

FIG. 4d depicts a further embodiment 403 of this invention in which particles 108 are linked together with linkers 110. Raman markers 112 are depicted associated with linkers 110 and receptors 124 are depicted attached to or associated with particles 108.

FIG. 4e depicts a yet further embodiment 404 of this invention, in which particles 108 are linked by linkers 110. Receptors 124 are associated with particles 108, and Raman markers 112 are depicted associated with receptors 124.

FIG. 4f depicts a still further embodiment 405 of this invention, wherein a plurality of particles 108 are linked via linkers 110. Receptors 124 are depicted associated with particles 108 having Raman markers 112 associated with receptors 124. Analyte moieties 128 are depicted in solution and associated with receptors 124.

It can be readily appreciated that one can make beads having Raman markers that identify any desired analyte. For example, a series of Raman markers may be used to label a series of beads and a series of analyte receptors may be specifically associated with a particular Raman maker combination. In such a fashion, a plurality of beads having Raman marker/analyte receptor pairs can be created. The use of such beads having Raman markers matched or “mapped” to particular analyte receptors can be very useful for detecting specific analytes in complex mixtures. Additionally, such paring of combinations of Raman markers with specific solid state syntheses can provide beads having a unique relationship of Raman marker to the synthesized chemical on the surface of the solid support, or bead.

### EXAMPLE 6

### Synthesis of Receptors of Nucleic Acid Oligomers

Thiol-derivatized DNA oligomers are synthesized by standard phosphoramidite chemistry according to the methods of Caruthers Gene Synthesis Machines: DNA Chemistry and Its Uses, Science 230:281-285 (1995), incorporated herein fully by reference. Such oligomers are obtained from Dr. Keith McKenney of The Institute for Genomic Research (TIGR), Rockville, Md., and are prepared according to the methods of Peterlinz et al. Observation of Hybridization and Dehybridization of Thiol-Tethered DNA Using Two-Color Surface Plasmon Resonance Spectroscopy, Journal American Chemical Society 119:3401-3402 (1997), incorporated herein fully by reference.

The DNA oligomers are selected to be in the range of about 10-50 bases in length, although much longer sequences can also be used. In other embodiments, the DNA oligomers are in the range of about 15-30 bases in length, and in alternative embodiments, the DNA oligomers are about 25 bases in length. If the oligomer is too long, the analyte molecule can be too far from the metal surface, and the surface enhancement of Raman resonance can be undesirably low. If the oligomer is too short, the specificity of hybridization can be too low. Therefore, the length of the oligomer is selected to optimize the sequence specificity and resonance enhancement of the analyte. In situations in which sequence specificity is less important than resonance enhancement, shorter oligomers can be desirable. Conversely, in situations in which a high degree of sequence specificity is desired, longer oligomers can be desirably used.

Two sets of complementary nucleotide oligomers are synthesized, one set being manufactured using moieties that lack a Raman active component. In certain embodiments, the DNA oligomer is synthesized using 2,6 di-aminopurine instead of adenine.

In other embodiments of this invention, peptide nucleic acid (“PNA”) receptors are used. Peptide nucleic acids have an affinity to RNA and DNA comparable to that of DNA, (Griffin (1998); Kyger et al (1998); Igloi (1998); Ratilainen et al. (1998), each reference herein incorporated fully by reference), and thus, can form hybridization pairs with mRNA. The difference between the chemical structures of PNA and DNA can result in a pronounced difference in their Raman spectra. In particular, the bands corresponding to nucleic acid phosphodiester backbone bonds, absent in the PNA attached to a substrate, appear when the PNA is bound to a DNA or mRNA ligand upon hybridization (Guan (1996)). PNA fragments can be obtained from Atom Sciences (Oak Ridge, Tenn.).

In further embodiments, receptors can be antibodies, antibody fragments, or other peptide or protein receptors. For example, cytokines or other specific hormones, neurotransmitters and the like can be specifically detected using the usual receptors for those molecules. Numerous recombinant and purified receptors for such biomolecules are known and can be advantageously used.

### EXAMPLE 7

### Linking of DNA to Colloidal Gold

The colloidal gold-coated quartz slides of Example 3 can then used as a matrix or substrate for the binding of DNA used for hybridization detection of analyte nucleic acids.

The gold colloid derivatized slides are placed in 1.0 M KH₂PO₄ buffer solution, pH 3.8, containing 1.0 μM thiol-derivatized DNA for a specific amount of time to achieve thiol-tethering of DNA. The surface is then passivated by exposing the DNA tethered slides to 1.0 mM mercaptohexanol (HS(CH₂)₆OH) for 1 hour. This treatment eliminates nonspecific binding of polynucleotides. Thorough rinsing with deionized water is required before analysis of hybridization.

C. Attachment of Markers to Resonance Domains

In certain embodiments of this invention, Raman markers maybe attached randomly to the enhancing particles. However, in other embodiments of this invention, markers can be localized to resonance domains within particle structures. Upon illumination of the particle structures, resonant domains can be heated, and that heating can cause partial melting of the metal particles. Typically, the dimensions of resonance domains are smaller than the wavelength of the incident light. The size of the resonance domains generated at certain wavelengths of incident light can be on the order of 1/25 of the wavelength of the light used in their generation. However, as the wavelength of light becomes longer, the size of the resonance domains can become smaller. Resonant domains are areas that can exhibit intense resonance, and can produce greater amplification of Raman signals than that possible in unaggregated metal or metal colloid substrates. Thus, resonance domains that are especially use ful for this invention can be made using incident light, which can result in resonance domains comprising between about 4 to about 10 monomer particles.

In certain embodiments of this invention, the property of particle structures to become locally heated can be used advantageously to localize marker molecules to those locations. To manufacture a particle structures having localization of resonance domain-specific markers, a surface containing particle structures is prepared as above. A solution containing receptor molecules is then placed on the surface and in contact with the particle structures. Pulses of laser light are used to illuminate the surface, and at those locations where resonance domains are created, the local temperature of the reaction mixture can reach the threshold for the formation of intermolecular bonds between the particle structures and the receptor, thus attaching the receptor to the particle structures. In general, any thermosensitive chemistry for linking the markers to the substrate can be used.

Generally, the power required to initiate marker molecule derivatization is less than that needed for photoaggregation. It can be desirable to provide temperatures at the resonance domains in the range of about 0° C. to about 500° C., alternatively in a range of about 20° C. to about 300° C., in other embodiments, in the range of about 50° C. to about 180° C. In yet other embodiments, the temperature can be in the range of about 70° C. to about 100° C.

The temperature needed will vary with the threshold temperature required to initiate the linkage of the receptor to the metal surface. In certain embodiments, it is desirable that the temperature locally at the resonance domains remain below the temperature at which bond breakage and reversal of the bond between the receptor and the metal surface occurs.

In other embodiments of this invention, photosensitive reagents can be used to link the marker to the particle structures or to linker molecules at specific locations. A number of such reagents can be obtained from Pierce Products Inc., Rockford, Ill. By the use of different photochemical linking agents, one can link different types of receptors to the same substrate. For example, one can attach DNA and proteins to the same substrate.

It can be desirable to limit the attachment of marker molecules to specific sites on an enhancing particle. This can be accomplished by using wavelengths of light that are relatively short, for example, less than about 1000 nm, in other embodiments, below about 600 nm, in yet other embodiments, below about 400 nm. Also, laser light can be desirable in situations in which the site of attachment is to be localized to areas of high electric field. In this case, it can be desirable to use double- or triple-photon processes, in which multiple photons having long wavelengths can reach the photoreactive moiety on the marker and particle structure to provide sufficient energy to cause a linking reaction to occur. This can occur even if the energy of a single photon is insufficient to initiate the photochemical reaction.

Once manufactured, marker molecules localized to the resonance domains of the particle pairs or fractal arrays can remain at those locations during subsequent exposures to incident light.

In other embodiments of this invention, attachment of markers at resonance domains can be performed using a scanning atomic force microscope (see Hansen et al. “A Technique for Positioning Nanoparticles Using an Atomic Force Microscope”, Nanotechnology 9:337-342 (1998), incorporated herein fully by reference). having a capillary tip and optical feedback. In these embodiments, the capillary contains derivatized markers which can be deposited onto a surface. In the process of deposition, the surface can be illuminated by incident electromagnetic radiation produced by a laser. At resonance domains, the resonance increases the intensity of the emitted radiation and thereby provides a signal to the optical feedback device to initiate deposition of markers at those locations, depending upon the intensity of electromagnetic radiation emitted from the surface in response to external illumination provided by the laser.

V. Detection of Analytes Using Raman Marked Beads

Detection of analytes according to methods of this invention includes the use of beads prepared according to the descriptions herein and a reader. Detection can be performed using a pre-manufactured substrate having wells or cells thereon, each having a single type of bead placed therein.

In general, an assay can comprise adding one or more types of beads of this invention to a solution containing an analyte or mixture of analytes. The analytes are permitted to bind to the receptors on the beads. Then, excess analyte is removed by washing or other method, unbound analytes are removed, leaving only those analytes having sufficient affinity for the receptor to remain bound. It can be readily appreciated that the amount and type of analytes bound to a given receptor type can depend on many variables, including the solvent conditions, pH, ionic concentration, temperature, the presence of blockers of non-specific bind, the presence of analytes similar to the one desired to be assayed, and other factors known in the art. After non-specific binding is reduced to a desired level, the beads can then be read. In certain embodiments, the beads can be placed into a reader matrix, which may comprise a multiwell plate, or the beads may remain in solution for analysis by a cell sorter or cell analyzer, wherein the beads replace by cells typically assayed. When a multiwell plate is used, a single bead maybe placed in the well, and the Raman signal from the bead determined using a Raman microscope or other miniaturized device. For some situations, it can be desirable to use a well having special optical characteristics that can increase the sensitivity of detection. Such devices are described in the U.S. patent application Ser. No. 09/669,369, incorporated herein fully by reference.

The matrix array can then be subjected to analysis using a reader or be performed using a light source focused upon the array, one cell at a time. Light is projected at the cell, and reflected, scattered, or re-emitted light can be collected and transmitted to the light detector. Collected light can be analyzed for Raman spectral features, and such features can be compared with Raman features derived from the Raman markers used.

Such known spectra can be imported from external databases, which can include information on biological significance of specific analytes. Analysis of information can be performed using a computer, which can be associated with a memory device for storing a program to carry out spectral analyses. Also, an output device, such as a screen display or a printer can provide information to the user. Such comparison can be the basis for determining the amount of analyte in the cell on the matrix array. Additionally, changes in the analyte due to the conditions of measurement can be determined, and any artifacts, such as non-specific binding so introduced can be discovered.

In yet other embodiments, a Raman array reader can be used to detect and quantify the amount of analyte bound to a cell of a matrix array. A Raman reader can be used for parallel, rapid and sensitive detection of analytes by acquiring Raman spectral features of each cell of an array and comparing the spectral features with known spectral features. Thus, the existence, identity and amount of a Raman marker can be determined.

In some embodiments, it can be desirable to use light sources that provide different wavelengths of light simultaneously. These sources can be less expensive and if the wavelengths are sufficiently different from each other, the interference with acquiring unique Raman spectra can be minimized.

A. Specificity of Ligand-Receptor Binding

The level of specificity of an assay of this invention can depend on the purposes of the assay. For example, if the aim of the assay is the detection of any of a series of related nucleotide sequences, herein termed “homologues,” the fidelity of the hybridization reaction need not be as high as an assay in which the detection and identification of single nucleotide polymorphisms (“SNPs”). The methods and compositions of this invention are well suited to detecting the presence or absence of a Raman band within a particular cell of a matrix array. Moreover, because the intensity of a characteristic Raman band is increased as the number of bound analytes increases, the methods of this invention can be used to quantify the amounts of analytes in an assay.

In general, the specificity of nucleotide-nucleotide hybridization reactions can depend on the conditions of hybridizations, herein termed “stringency.” Hybridization conditions are described in Sambrook et al., Molecular Cloning: A Laboratory Manual, Second Edition, Cold Springs Harbor Laboratory Press (1989), incorporated herein fully by reference. In general, as used herein, the term “high stringency” refers to conditions in which the temperature of hybridization is about 5° C. to about 10° C. below the melting temperature of the duplex. The melting temperature_{TM} of an oligonucleotide duplex can be estimated as follows:

Tₘ=81.5-16.6(log₁₀[Na⁺])+0.41 (fraction C+G)−(600/N),

where [Na⁺] is the sodium concentration, C+G is the amount of cytosine (C) and guanine (G) as a fraction of the total number of nucleotide bases, and N is the chain length. High stringency involves either the incubation of or the washing of ligand and receptor nucleotides under conditions that disfavor hybridization of non-complementary sequences. Such conditions include the use of high temperatures, low salt concentration and high detergent concentrations. Using high stringency, detection of sequences having only one non-complementary base (one “mismatch”) can be accomplished. Conversely, low stringency conditions include lower temperatures, higher salt concentrations and lower concentrations of detergents. Low stringency conditions can be especially desirable if the purpose of the assay is the detection of homologues, in which base-pair mismatches are present.

Moreover, in certain embodiments of this invention, one can obtain qualitative information regarding the number of base-pair mismatches by making repeated spectroscopic measurements of the same cell under progressively higher stringency conditions. For example, if an analyte has a relatively large number of mismatches, so that a detectable signal is present only after low stringency washing, subsequent washing of the same cell at high stringency conditions can remove the analyte from that cell. This stringency is herein termed the “stringencythreshold.” By comparing the number of mismatches with the stringency threshold, one can determine the relative degrees of homology of nucleic acid sequences without determining the actual sequences.

The specificity of binding of analytes is often not perfect, especially when antibodies, native or recombinant receptors, lectins and the like are used. Antibodies can bind other analytes non-specifically, in addition to their direct targets. In such situations, spectral analysis of Raman spectral features can permit discrimination and quantitation of the desired analyte even in the presence of non-specific binding. Native receptors including cytokine receptors (e.g., IL2, IL4, TNF alpha, and the like), hormone receptors (e.g., for insulin, glucagon, pituitary hormones and the like), neurotransmitter receptors (e.g., acetylcholine, norepinephrine, substance P, Vasoactive Intestinal Peptide, and the like), metabolite uptake carriers (e.g., glucose transporters, amino acid transporters, vitamin transporters and the like), and the like also may bind with different affinities to different potential analytes.

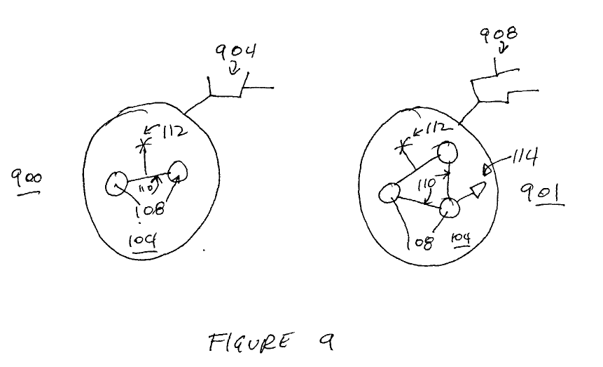

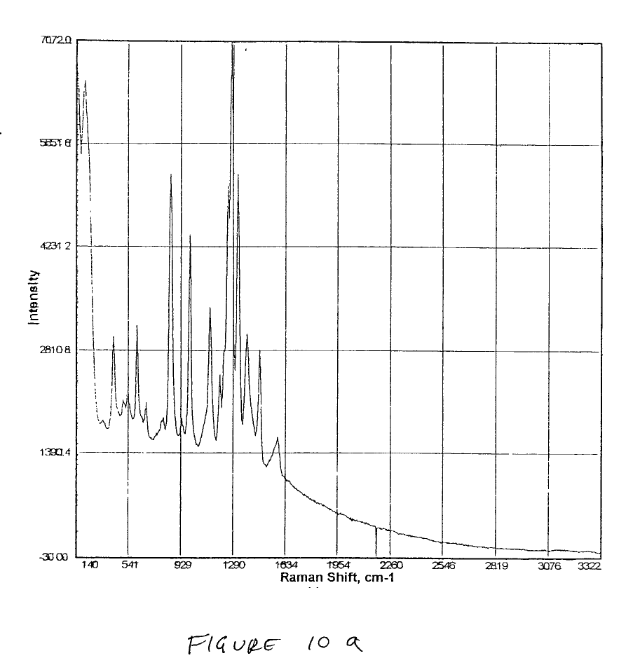

FIG. 5a depicts an embodiment of this invention 500 comprising a bead matrix 104 having a single enhancing particle 108 therein. Raman markers 112 are depicted attached to particle 108. Receptors 124 are depicted on the surface of the bead and analytes 128 are depicted associated with receptors 124 and free in solution.

FIG. 5b depicts an alternative embodiment 501 of this invention, wherein a bead matrix 104 has a pair of particles 108 joined by linker 110. Raman markers 112 are shown associated with the linker 110. The Raman markers 112 may be different moieties attached to linker 110, or may be the linker molecule 110. Receptors 124 are shown on the surface of bead matrix 110, and analytes 128 are depicted associated with receptors 124 and free in solution.

FIG. 5c depicts an alternative embodiment 502 of this invention, wherein the bead matrix 104 has a plurality of particles 108 linked by linkers 110. Raman markers 112 are shown associated with particles 108. Receptors 124 are shown on the surface of the bead matrix 104 and analytes 128 are depicted associated with receptors 124 and free in solution.

FIG. 6 depicts a pair of particles 603 and 602, each comprising a bead matrix 104, and structures of enhancing particles therein. Bead 602 has a plurality of linked particles 108, linkers 110 and receptors 112 (*) and 113 (+) and 114 (Δ) are depicted attached to either linkers 110 or to particles 108. In contrast, bead 603 has only one type of Raman marker 112 (*). Thus, beads 602 and 603 will exhibit different patterns of Raman signals.

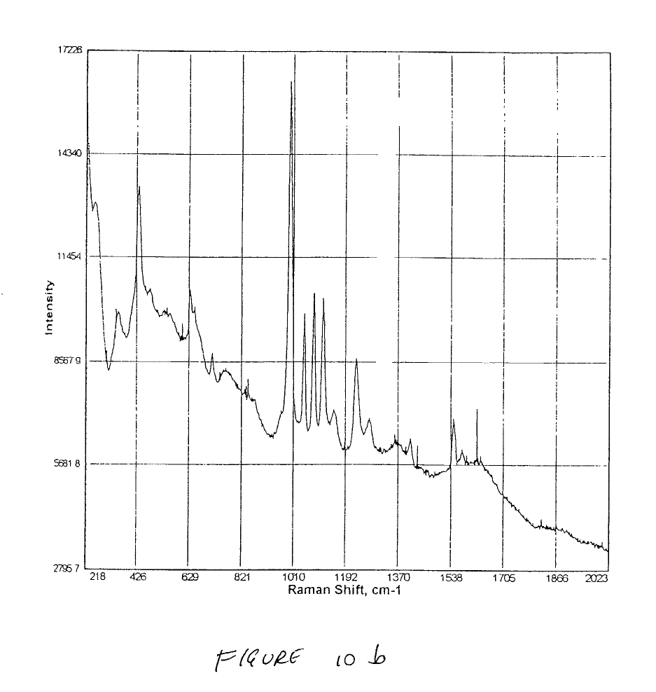

FIG. 7 depicts a biochip 700 of this invention having a substrate 704 having a surface 708 thereon. Beads 602 and 603 are depicted on surface 708. Additional beads 610a-610d, each having unique combinations of Raman markers, are also shown.

It can be appreciated that detection and identification of the beads depicted can be carried out using Raman detectors. Once a bead is identified by its combination of Raman markers, and the analyte bound to the bead is detected, one can readily conclude the identify of the analyte by reference to the combination of Raman markers present on the bead.

B., Detection of Markers By Raman Spectroscopy

1. Raman Spectroscopy

Devices used to perform analyses of Raman markers according to the methods of this invention can include any device that can produce laser light of the wavelengths needed for analysis. For example, the T64000 Raman Spectrometer (The Ultimate Raman Spectrometer Instruments S. A. Ltd. (UK) can be used. Desirable features of a suitable instrument include the ability to position the sample compartment to adjust the sensitivity of the spectrum, provides for low frequency measurements, provides adequate spectral resolution, and a liquid nitrogen cooled charged coupled device (“CCD”) detector. The spectrometer is suitably equipped with a laser light source comprising a continuous wave, frequency doubled argon laser. Because the purine and pyrimidine ring structures of nucleotides have characteristic absorption maxima in the ultraviolet range, it can be desirable to provide laser light having emission wavelengths in the ultraviolet range. A suitable laser is the Inova 300 FReD, available from Coherent Inc., Santa Clara, Calif. Laser power for certain embodiments of this invention can be maintained at about 5 milliwatts at 257 nm, or 1 milliwatt at 244 nm, 229 nm and 238 nm.

For other applications, it can be desirable to use longer wavelengths, for example, in the range of about 830 nm. Such a light source is a continuous-wave titanium:sapphire laser. For other applications, light in the visible range can be suitable To detect analytes in a single cell, it can be desirable to provide Raman spectroscopic measurements over areas that are sufficiently small to avoid cross-readings from adjacent cells. For matrix arrays having 100 μm×100 μm per side, it is desirable to provide a narrow, focused beam of incident light. By way of example, a Raman reader suitable for analyte detection according to this invention is described in U.S. patent application Ser. No. 09/939,887, incorporated herein fully by reference.

C. Detection of Analytes by Fluorescence

Many methods exist for detection and quantification of analytes using fluorescent labels. For example, one may attach fluorescein to a series of analytes in a solution, add a mixture of Raman marked beads having analyte receptors, and permit the fluorescently labeled analytes to bind to the beads bearing receptors specific for that analyte. The beads can be separated and the Raman signals analyzed to determine the type of analyte receptor present on the bead.

VI. Use of Raman Marked Biomarkers

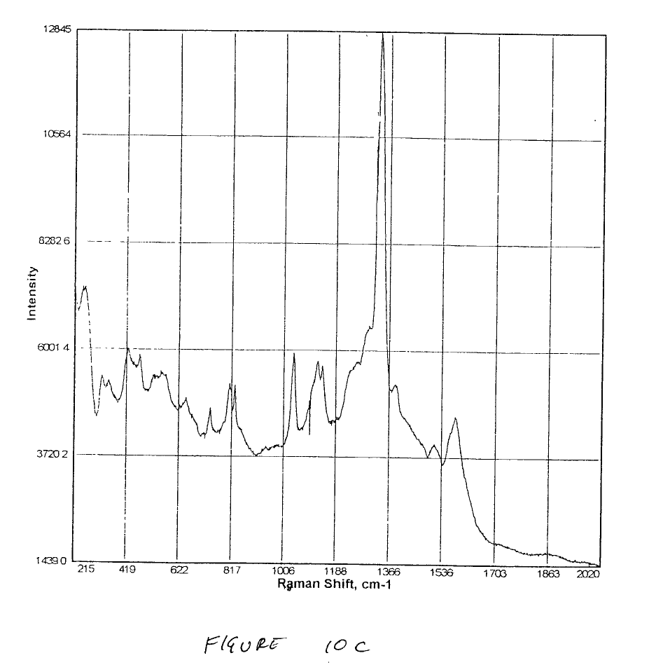

An additional use of the Raman marked beads of this invention is for identification of cells, tissues, and pathogens in animals, plants or other organisms. For example, FIG. 8a depicts a series of beads that are labeled with receptors specific for cell, tissue and pathogen receptors. Bead 602 is depicted having a receptor specific for muscle cells (M), bead 603 has a receptor specific for connective tissue (CON), bead 604 has a receptor specific for blood vessels (BV), bead 605 has a receptor specific for glands (G), bead 606 has a receptor specific for cancer (CA), bead 607 has a receptor specific for a virus (V), bead 608 has a receptor specific for a bacterium (B), and bead 609 has a receptor specific for a specific blood cell (BC).

FIG. 8b depicts a schematic representation of a tissue section 801 having a plurality of different tissues shown. Muscle tissue 804 has beads 602 attached thereto, connective tissue 808 has beads 603 attached thereto, blood vessel 812 has beads 604 attached thereto, gland 816 has beads 605 attached thereto, an area of viral infection 820 has beads 607 attached thereto, cancer tissue 824 has beads 606 attached thereto, area of bacterial infection 828 has beads 608 attached thereto, and blood cell 832 has beads 609 attached thereto. Thus, identification of cell, tissue and/or pathogen type need not be made based on cell or tissue morphology alone, but can also be made using specific biomarkers of this invention.

VII. Use of Raman Marked Beads in Combinatorial Synthesis

In alternative uses of the Raman marked beads of this invention, the beads can be used as solid supports for chemical syntheses. It can be appreciated that combinatorial chemistry can be made easier by simple methods of tagging or identifying individual beads subject to “split and pool” synthetic methods. For example, a series of Raman marked beads can be prepared, and individual syntheses may be carried out on individual beads.

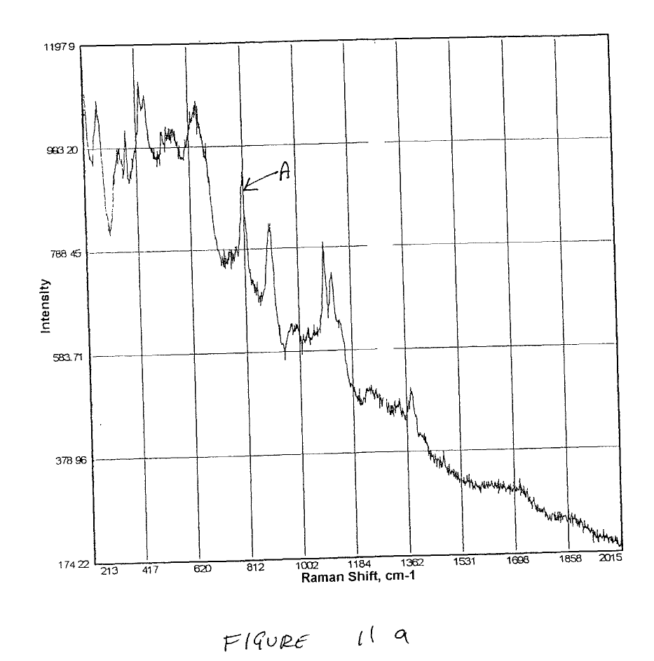

FIG. 9 depicts two beads 900 and 901 having enhancing particle groups therein. Bead 901 has three particles 108 linked by linkers 110 and having Raman markers 112 and 114 thereon. Bead 901 has synthesized chemical 908 on the surface of bead matrix 104. Bead 900 has two particles 108 linked by linker 110 having Raman marker 112 attached thereto. Chemical 904 is shown on the surface of bead matrix 104.

VIII. Simultaneous Detection of Different Raman Markers

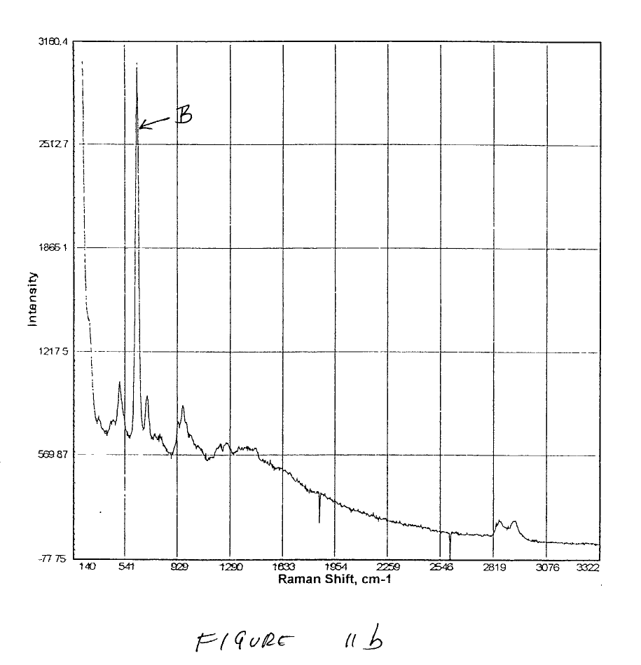

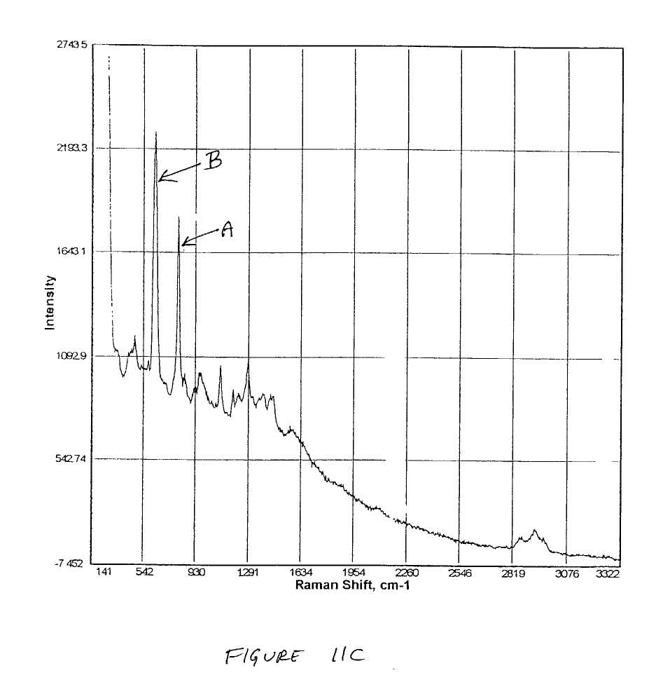

FIG. 10a depicts a Raman spectrum of mercaptopurine. Fifty μL of a 10⁻³ M solution of mercaptopurine was added to 50 μL of Ag aggregates made according to this invention and treated with Na citrate and 0.2 M NaCl to form colloidal silver aggregates. The mixture was placed on aluminum foil, and the Raman spectrum obtained.

FIG. 10b depicts a Raman spectrum of DTP. Fifty μL of a 1:100 dilution of a saturated solution of DTP in water was added to Fifty μL of a colloidal silver aggregate as described for FIG. 10a. The solution was placed on aluminum surface and the Raman spectrum was obtained.

FIG. 10c depicts a Raman spectrum of dinitrophenol (DNP) derivatized cystine. Fifty μL of a silver colloidal aggregate as described above was added to 2 mM DNP-cystine in 0.3 M NaOH. The mixture was placed on aluminum and the Raman spectrum obtained.

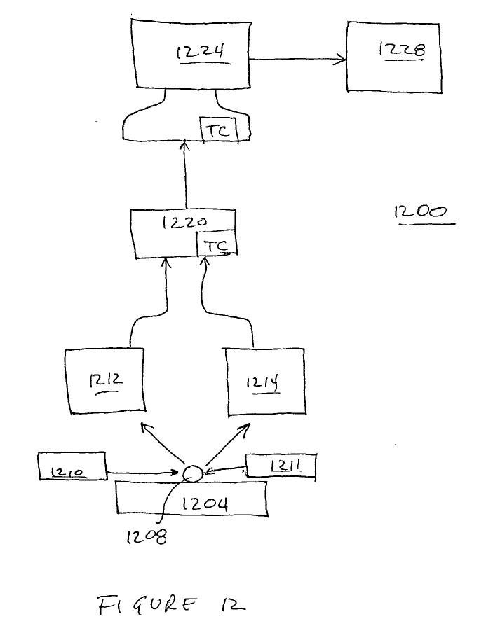

FIG. 11a depicts the Raman spectrum of purine. Fifty μL of a 10⁻³ M solution of purine was added to Fifty μL of a colloidal silver aggregate, the mixture placed on aluminum, and the Raman spectrum was obtained. Note peak “A” in the trace.

FIG. 11b depicts the Raman spectrum of mercaptoethylether (MEE). Fifty μL of colloidal silver aggregates were mixed with Fifty μL of a 10⁻⁵ M solution of MEE. The Raman spectrum was obtained. Note peak “B” in the trace.

FIG. 11c depicts the Raman spectra obtained for a mixture of MEE and purine together. Note that peaks “A” and “B” are separated from each other.

Thus, one can use a multiplicity of different Raman markers simultaneously to identify a large number of different combinations.

Systems for analysis of data obtained from Raman readers and/or analyte detectors can be analyzed using systems. In certain systems, a computer having trusted computing space can be used to provide control over access to information obtained using the detection methods of this invention.

FIG. 12 depicts a schematic drawing of a system 1200 for data analysis. Chip 1204 has bead 1208 thereon having an enhancing structure, a unique combination of Raman markers and an analyte receptor type associated with the unique combination of Raman markers. Incident electromagnetic radiation generated by a Raman illuminator 1210 produce Raman signals that are detected by Raman reader 1212. Incident electromagnetic radiation generated by analyte detector 1211 produce analyte-specific signals that are detected by analyte reader 1214. Signals from Raman reader 1212 and analyte detector 1214 are received by storage device 1220 having trusted computing space therein (TC). Information stored in storage device 1220 is sent to computer 1224 having trusted computing space (TC) therein. Signals from Raman reader 1212 and analyte detector 1214 are compared and mapped to each other to provide identification of the analyte detected. Output from computer 1224 is provided to output device 1228 for display.

### INDUSTRIAL APPLICABILITY

The particle structures of this invention can be used in the fields of chemistry and biotechnology for the detection of analytes in complex solutions containing many different species of molecules. Additionally, the methods of this invention can be used for the detection and quantification of analytes using spectroscopic methods, including fluorescence spectroscopy, immunobiology and mass spectroscopy.

We claim:

1. A bead comprising:
a non-spherical enhancing particle; and
a Raman marker attached thereto,
wherein said enhancing particle and said Raman marker are within the bead.

2. The bead of claim 1, wherein said non-spherical enhancing particle is rod-shaped or crystalline.

3. The bead of claim 1, further comprising at least one receptor on the outside of said bead.

4. A bead comprising:
a spherical enhancing particle having a Raman marker attached thereto and inside a bead; and
at least one receptor attached to the outer surface of said bead.

5. A bead comprising:
a plurality of enhancing particles having at least one Raman marker associated with at least one of said enhancing particles, said plurality of particles within said bead; and
at least one receptor attached to the outer surface of said bead.

6. An enhancing particle structure, comprising:
at least two particles;
at least one linker; and
at least one Raman marker, wherein said linker links said at least two particles together and said Raman marker is associated with said linker.

7. The enhancing particle structure of claim 6, wherein said at least one Raman marker is associated with at least one of said particles.

8. The enhancing particle structure of claim 6, further comprising at least one receptor.

9. The enhancing particle structure of claim 8, wherein said at least one receptor is associated with a linker or a particle.

10. The enhancing particle structure of claim 9, wherein said Raman marker is associated with said at least one receptor.

11. The enhancing particle structure of claim 8, where an analyte is associated with said at least one receptor.

12. The bead of claim 4, wherein an analyte is associated with said at least one receptor.

13. The enhancing particle structure of claim 6 associated with a bead, and at least one analyte receptor associated with an outer surface of said bead, thereby forming an analyte receptor-bead complex.

14. The complex of claim 13, wherein an analyte is associated with said at least one receptor.

15. A mixture, comprising:
a plurality of beads, each bead of said plurality having an enhancing particle structure associated therewith, and each enhancing particle structure having a combination of Raman markers different from Raman markers associated with all other of said plurality of beads.

16. A biochip, comprising:
a substrate;
at least one bead having an enhancing particle structure with an identifying combination of Raman markers thereon; and
at least one receptor associated with at least one of said beads.

17. A method for detecting an analyte, comprising:
(a) providing at least one bead having an enhancing particle structure with a characteristic Raman marker associated therewith and at least one analyte receptor;
(b) providing a sample containing an analyte;
(c) permitting said analyte to associate with said receptor;
(d) detecting a Raman signal from said Raman marker; and
(e) detecting a signal from said analyte.

18. The method of claim 17, wherein said Raman marker and said analyte are sufficiently isolated from each other so that substantially no quenching of said signal from said analyte occurs.

19. The method of claim 17, further comprising the step of associating said signal of said Raman marker from said signal of said analyte.

20. The method of claim 17, wherein step (a) provides a plurality of beads, each having a unique combination of Raman markers, and each bead having a unique receptor type, and wherein step (b) provides a plurality of analytes.

21. A method for identifying a structure within a biological sample, comprising:
(a) providing at least one enhancing particle structure associated with a Raman marker and a receptor;
(b) exposing said biological sample to said at least one enhancing particle structure;
(c) obtaining a Raman signal from said Raman marker; and
(d) associating said Raman signal with a structure in said biological sample.

22. The method of claim 21, wherein said structure is selected from the group consisting of cells, tissues and pathogens.

23. The method of claim 22, wherein said cell is selected from the group consisting of muscle cells, connective tissue cells, blood vessel cells, gland cells, cancer cells, and blood cells.

24. The method of claim 22, wherein said tissue is selected from the group consisting of extracellular proteins, extracellular lipids, extracellular carbohydrates, and combinations thereof.

25. A bead, comprising:
an outer surface;
an enhancing particle structure associated with at least one Raman marker, said enhancing particle structure being within said bead; and
a synthesized chemical on the outer surface of said bead.

26. A system for detecting an analyte associated with a bead, comprising:
at least one bead having:
an enhancing particle structure having a Raman marker;
said enhancing particle structure within said bead;
at least one analyte receptor; and
an analyte associated with said analyte receptor;
a Raman reader; and
means for detecting said analyte.

27. The method of claim 26, wherein said means for detecting said analyte comprises a fluorescence method.

28. The system of claim 26, further comprising a computer for analyzing a signal provided by said Raman reader and a signal provided by said means for detecting said analyte.

32. A system for analyte detection, comprising:
a substrate having a plurality of beads thereon, each of said beads having:
an enhancing particle structure having a unique combination of Raman markers; and
at least one analyte receptor associated with said bead;
a Raman reader;
an analyte detector associated with each of said beads;
means for mapping a signal received from said Raman reader to said analyte receptors associated with each unique combination of Raman markers; and
an output device.

33. The system of claim 32, further comprising a memory storage device.

34. The system of claim 32, having at least one trusted computing space.

35. The system of claim 33, having at least one trusted computing space.
